(19) United States
(12) Patent Application Publication
(10) Pub. No.: US 2013/0253048 A1
(43) Pub. Date: Sep. 26, 2013

(54) Pharmaceutical Composition for Promoting Osteogenesis Containing Axial-equatorial Aryl-oriented Furofuran-type Lignan, and Pharmaceutical Preparation, Functional Food Product, and Health Food Product Comprising Composition

(71) Applicant: ERINA CO., INC., Tokyo (JP)

(72) Inventors: Midori ASAI, Tajimi-shi (JP); Young-Sil Lee, Kasugai-shi (JP); Naomi MASE, Chiryu-shi (JP); Bong-Keun CHOI, Kasugai-shi (JP); Byung-Yoon CHA, Kasugai-shi (JP); Takayuki YONEZAWA, Tokyo (JP); Toshiaki TERUYA, Urasoe-shi (JP); Kazuo NAGAI, Tokyo (JP); Je-Tae WOO, Tokyo (JP)

(73) Assignee: ERINA CO., INC., Tokyo (JP)

(21) Appl. No.: 13/739,340

(22) Filed: Jan. 11, 2013

Related U.S. Application Data
(63) Continuation of application No. PCT/JP2011/065912, filed on Jul. 12, 2011.

(30) Foreign Application Priority Data
Jul. 12, 2010 (JP) JP2010-157598

(51) Int. Cl. A61K 31/36; A23L 1/30

(52) U.S. Cl. CPC A61K 31/36; A23L 1/3002
USPC 514/464; 549/435

(57) ABSTRACT

Provided is a composition that, even when used in a very small amount, has the effect of improving bone density and promoting bone growth, and has few side effects, and also provided are a pharmaceutical preparation for promoting bone growth, functional food product, and health food product containing the composition as an active ingredient. The composition can be obtained from any plant part selected from the group consisting of the bud, leaf, bark or wood of a plant belonging to the family Magnoliaceae. The composition comprises at least one selected from the group consisting of fargesin and physiologically acceptable salts, hydrates, and glycosides thereof. The pharmaceutical preparation, functional food product, and health food product comprising this composition as an active ingredient sufficiently improve bone density and promote bone growth, even when used in small amounts, and therefore realize preventive and/or therapeutic effects against bone disease etc. such as osteoporosis.

### TECHNICAL FIELD

The present invention relates to pharmaceutical composition for osteogenesis promotion comprising axial-equatorial aryl orientational furofuran type lignan, pharmaceutical preparation comprising thereof, functional food comprising thereof and health food comprising thereof. Particularly, the present invention relates to pharmaceutical composition for osteogenesis promotion comprising Fargesin and its derivatives, pharmaceutical preparation comprising thereof, functional food comprising thereof and health food comprising thereof.

### BACKGROUND ART

Recently, bone disease patient number of aged people is increasing, depending on the increase of a mean age. Here, the term “bone disease” includes non-metabolic bone disease such as bone fracture and the like, and metabolic bone disease such as osteoporosis, bone Paget's disease, osteomalacia and the like. Several bone disease are caused by an inflammatory arthritis such as osteoarthritis, rheumatoid arthritis, and the like. Rheumatoid arthritis may cause periarticular site of osteoporosis.

The metabolic bone disease, osteoporosis, is roughly classified into primary osteoporosis, which is not caused by other disease, and secondary osteoporosis, which is caused by other disease such as malignancy, rheumatoid, and others. In the osteoporosis, the primary osteoporosis accounts for 95% of entire of osteoporosis. Further, osteoporosis has type I of which rate of crises is 6 times higher in women than men, and type II which generally develops in the patient over 60 years old.

In Type I osteoporosis, osteoclast cells, which plays a role in the bone metabolism, is activated. As a result, bone resorption is enhanced, and bone density is reduced. This is caused by increase of cytokine levels derived from decreased estrogen secretion from ovary. Therefore, β-estradiol, a kind of estrogen, is uses as a prophylaxis and/or treatment agent for osteoporosis.

The bone fracture as the non-metabolic bone disease is resulting from the large force loading onto the bone having normal strength at one stroke in a healthy person. In contrast, it is resulting from less force loading, which does not cause the bone fracture in the healthy person; onto the bones weaken by cancer, osteoporosis and the like. It is referred to as pathological fracture.

The bone fracture is caused by repeated load onto the same place from exercise. It is referred to as “stress fracture”. It is said that the stress fracture is sometimes developed on metatarsal bone. As compared to men athletes, women athletes prone to breaking bones than men. As one of the reasons, there is mentioned that incidence rate of osteoporosis is higher in the women athletes than men athletes.

An index of the metabolic bone disease, abnormal blood calcium level is employed, because released amount of calcium into the blood is increasing after the development of osteoporosis.

Among the bone disease described above, in order to treat the metabolic bone disease except the bone fracture, active vitamin D₃ as a derivative of vitamin D which plays an important role in calcium metabolism, calcitonin and derivative thereof, hormonal agent such as β estradiol and the like, and a variety of calcium compound and preparations are clinically used. It is known that among them, vitamin D₃ approaches the osteoclast cells and osteoblast cells, proto cells or precursor cells thereof to promote their proliferation.

It is not a treatment agent, it is known that fargesin having the following chemical structure (Fargesin; MW=370.4) dose-dependently inhibits tartrate-resistant acid phosphatase activities in mouse monocyte macrophage type cells under RANKL stimulation in the culture, or marrow monocytoid cells in the culture, and it also inhibits the phosphorylation of p38 and I-κB by RANKL stimulation (non-patent document 1). Fargesin is a lignan, and it is classified as the axial-equatorial aryl orientational furofuran type one based on its structure (patent document 2). Fargesin described on the reference is extracted from M. fargesii belonging to Magnoliaceae or Thurber's Magnolia (Magnolia kobus DC. var. borealis Sarg.).

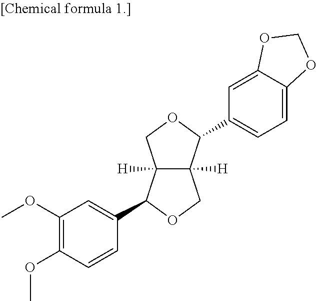 [SMILES: COc1ccc([C@@H]2OC[C@H]3[C@@H]2CO[C@@H]3c2ccc3c(c2)OCO3)cc1OC]

### PRIOR ART REFERENCE

### Patent Document

[Patent document 1] WO 1990/013299 A1
[Patent document 2] JP 2003-522787

### Non-Patent Document

[Non-patent document 1] Proceedings of annual meeting of the Japanese Society of Pharmacognosy, vol. 55, p. 212, Compounds inhibits osteoclast cell proliferation derived from Magnolis, Naomi MASE, Bong-Kuen CHOI, Morikazu HASEGAWA, Toshiaki TERUYA, Takayuki YONEZAWA, Byung-Yoon CHA, Kazuo NAGAI, Je-Tae WOO
[Non-patent document 2] RESEARCH BULLETIN OF HOKKAIDO UNIVERSITY FORESTS, 53(1): 1-28 Extractives of kitakobushi Magnolia kobus DC. Var. borealis Sarg. I, Yun-Geun KIM, Shuji OZAWA, Yoshihiro SANO, and Takashi SASAYA

### SUMMARY OF THE INVENTION

### Problem to be Solved by the Invention

Calcitonin and the derivative thereof, the hormone agent such as β-estradiol and the like are used for treating the bone disease. However, these agents sometimes cannot to be administrated to the patient, depending on absorption in vivo or metabolic problem. Also, there is the problem that it lacks predictability, because their receptor levels are highly individual.

Therefore, there is a need for new preparation to complement the prescription for treating the bone disease, and for new treatment method by using such agents. Also, there is a need for prophylaxis agent to reverse the progress of osteoporosis.

Since bone strength is increased by exercise load, the common exercise is effective for preventing osteoporosis. However, if an aged person or a person being usually sedentary performs exercise with no warm up or vigorous exercise, they carry a risk to strain their body. Therefore, it is effective to perform the exercise and to take the functional food or health food. In these foods, even more than effectiveness, higher safety more than the pharmaceutical preparations should be saved. Because of such a situation, no side effects are more important. Therefore, ingredients included in them are preferable to produce the effect, if their amount is small.

The treatment of the bone fracture is performed by surgical operation or reset in principle, except administration of an analgesic agent. Since osteoporosis causes the bone fracture, the treatment of osteoporosis and prevention are necessary to prevent the bone fracture.

In order to prevent the stress fracture among the bone fracture, appropriate dietary control, training and organizing to take an adequate rest are necessary. Also, it is effective for the prevention of the stress fracture to take the functional foods or health foods to complement them. For these foods, it is important that they do not cause any side effects like those above. Therefore, the composition for them is preferable to show sufficient prophylactic effects in the small amount.

Accordingly, there are strong needs for osteogenesis improving pharmaceutical preparations having highly predictable effects, particularly, which has high improving effects for the cancellous bone. From the view point of preventive medicine, there is strong social need for the foods comprising the composition.

### Means for Solving the Problem

The first aspect of the present invention is a pharmaceutical composition for promoting osteogenesis comprising at least a substance selected from the group consisting of a axial-equatorial aryl orientation furofuran type lignin compound shown in the following chemical formula (I), a pharmacologically acceptable salt thereof, a pharmacologically acceptable hydrate thereof, and a pharmacologically acceptable glycoside thereof.

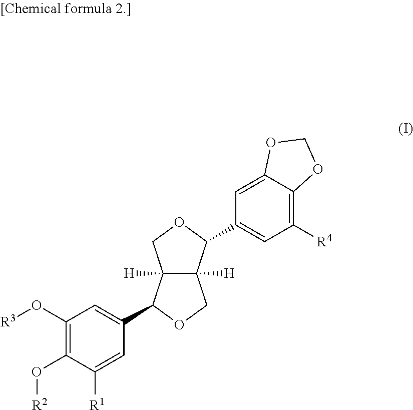

(In the formula, R¹ and R⁴ independently show one of functional group selected from the group consisting of a hydrogen atom, alkyl group having a carbon number 1 to 3, hydroxyl group, alkoxy group having the carbon number 1 to 3; R² and R³ independently show one of functional group selected from the group consisting of alkyl group having a carbon number 1 to 3.)

The pharmaceutical composition for promoting osteogenesis preferably comprises at least a substance selected from the group consisting of a compound shown in the following chemical formula (II), a pharmacologically acceptable salt thereof, a pharmacologically acceptable hydrate thereof, and a pharmacologically acceptable glycoside thereof.

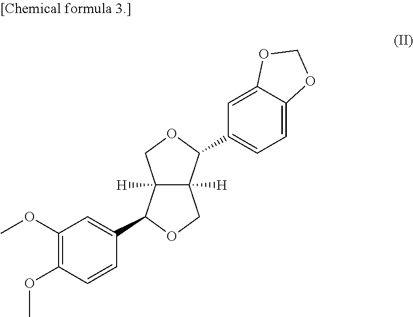 [SMILES: COc1ccc([C@@H]2OC[C@H]3[C@@H]2CO[C@@H]3c2ccc3c(c2)OCO3)cc1OC]

The pharmaceutical composition is preferably applied for osteoporosis, hypercalcemia, hyper-parathyroid hormonemia, bone Paget's disease, arthritis, rheumatoid arthritis, metastasis of mammary cancer, osteomalacia, malignancy, and nutrition disorder, traumatic bone fracture, stress fracture or the like. Particularly, it is preferably applied to osteoporosis.

The second aspect of the present invention is a pharmaceutical composition for promoting osteogenesis comprising an extract fraction obtained from one organ selected from the group consisting of a flower bud, leaf, cortex and xylem of Magnoliaceae plant, of which fraction containing the compound shown in the above-mentioned formula (II).

The organ selected from the group consisting of the flower bud, leaf, cortex and xylem of Magnoliaceae plant is preferably obtained from the plant selected from the group consisting of Tamushiba (Magnolia salicifolia Maximowicz), Kobushi (Magnolia kobus De Candolle, Magnolia biondii Pampanini, Magnolia sprengeri Pampanini), Hakumokuren (Magnolia heptapeta Dandy (Magnolia denudata Desrousseaux) (Magnoli-aceae), and Kitakobushi (Magnolia praecocissima var. borealis). When the flower bud is used of Kobushi, fractions obtained from it have higher fargesin content.

The third aspect of the present invention is the pharmaceutical preparation comprising the above-mentioned pharmaceutical composition as the active ingredient to be administrated at a predetermined dosage. In the pharmaceutical preparation, the predetermined dose is preferably 10 to 350 mg/day in compound equivalent, more preferably 20 to 175 mg/day. The pharmaceutical preparation is preferably applied for osteoporosis, hypercalcemia, hyper-parathyroid hormonemia, bone Paget's disease, arthritis, rheumatoid arthritis, metastasis of mammary cancer, osteomalacia, malignancy, and nutrition disorder, traumatic bone fracture, stress fracture or the like. Particularly, it is preferably applied to osteoporosis.

The fourth aspect of the present invention is the functional food comprising the composition of the first and/or the second aspect at the predetermined content. The fifth aspect of the present invention is the functional food comprising the composition of the first and/or the second aspect at the predetermined content.

The food is more preferably a functional food or the health food for promoting the osteogenesis. The predetermined content is preferably 1 to 1,000 mg/kg. The amount intake of the functional food or the health food is preferably 10 to 350 mg/day expressed in terms of the amount of the above-mentioned compound, and more preferably 20 to 175 mg/day. The functional food or the health food may be preferably used to the bone disease such as osteoporosis, more preferably used to osteoporosis.

The food may be cookies and biscuits, wheat and miscellaneous cereals for being supplemented to rice, noodles such as Japanese wheat noodle, soba noodle, and pasta, dairy product such as cheese, yogurt, jam, mayonnaise, processed soy product such as soybean paste, soy source, tea, coffee and cocoa, nonalcoholic beverage such as soft drinks and fruits juice, alcoholic beverage such as medicated liquor, snacks such as candy (drops), and chocolate, chewing gum, Japanese cracker, sweets made from azuki-bean such as azuki-bean jelly, to produce the functional food.

The sixth aspect of the present invention is a treatment method for osteogenesis comprising the step for non-parenterally or parenterally administrating any one selected from the group consisting of the composition of the first aspect of the present invention, that of the second aspect of the present invention, and the pharmaceutical preparations to the patient necessary for promoting the cortical bone formation or the cancellous bone formation.

Wherein, the composition or the pharmaceutical preparation is preferably taken orally; more preferably, it is combined with the exercise therapy, because fixing ration of calcium to the bone is improved.

Small amounts of the composition, pharmaceutical preparation or foods, or the active ingredients or compositions used in the treatment method have sufficient advantageous effects for the bone density enhancement or the bone growth acceleration, and another advantageous effect to prevent and/or treat the bone diseases. Therefore, they have few side effects.

### BRIEF DESCRIPTION OF DRAWINGS

FIG. 1 is a graph showing ¹H NMR specturm (400 MHz, CDCL₃) of fargesin.

FIG. 2 is the graph showing ¹³C NMR spectrum (400 MHz, CDCL₃) of fargesin.

FIG. 3A shows measurement site in a femur.

FIG. 3B shows a section of the measurement site in the femur.

FIG. 4 is the graph showing a bone density (mg/cm³) of the total bone, when a test substance is administrated to an ovariectomized mouse.

FIG. 5 is the graph showing a bone density (mg/cm³) of the cancellous bone, when a test substance is administrated to an ovariectomized mouse.

FIG. 6 is the graph showing a bone density (mg/cm³) of the cortical bone, when a test substance is administrated to an ovariectomized mouse.

FIG. 7 is an image of transmitted light of the bone (left column) and a fluorescence staining image of the bone by using calcein (right column).

FIG. 8A is the graph showing ALP activities and TRAP activities by using a relative ratio to a negative control (%), when the test substance is administrated to an ovariectomized mouse.

FIG. 8B is a double stained image of ALP and TRAP, when the substance is administrated to co-cultured cells.

FIG. 9A is the graph showing ALP activities and results of MTT assay by using the relative ratio the negative control (%), when the test substance is administrated to osteoblast-like cells.

FIG. 9B is the graph showing ALP stained image, when the test substance is administrated to the osteoblast-like cells.

FIG. 10A is the graph showing ALP activities by using the relative ratio the negative control (%), when the test substance is administrated to calcified osteoblast-like cells.

FIG. 10B is the graph showing ALP stained image, when the test substance is administrated to the calcified osteoblast-like cells.

FIG. 10C is the graph showing stained image of mineral deposition, when the test substance is administrated to the osteoblast-like cells.

FIG. 11A is the graph showing the bone density in the mice of each group, a pseudo-operation group (Sham), or an ovariectomized group (OVX), after 3 month from the administration, when each substance was administrated to the mice.

FIG. 11B is a figure showing the cancellous bone density of mice in the groups in shown in FIG. 11A.

FIG. 12 is a figure showing polar coordination strength of mouse femur in the groups shown in FIG. 11A.

FIG. 13 is a figure showing values TRACP5b of mouse sera of the groups shown in FIG. 11A.

FIG. 14A is a graph showing the total bone density of the mice of the sRANKL administration or the test sample administration group after 13 days from administration.

FIG. 14B is the graph showing the mouse cancellous bone density of the group shown in FIG. 14A.

FIG. 15 is the graph showing the polar coordination strength of mouse femur in the groups shown in FIG. 14A.

### MODE FOR CARRYING OUT THE INVENTION

The present invention is explained in detail hereinbelow.

The first aspect of the present invention is a pharmaceutical composition for promoting osteogenesis comprising at least a compound shown in the following formula (I), the axial-equatorial aryl orientational furofuran type lignin compound and their derivatives.

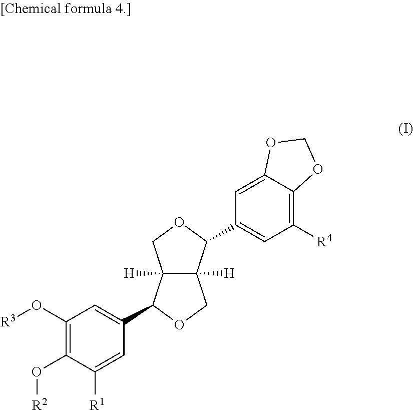

In the formula, R¹ and R⁴ independently show one of functional group selected from the group consisting of a hydrogen atom, alkyl group having a carbon number 1 to 3, hydroxyl group, alkoxy group having the carbon number 1 to 3; R² and R³ independently show one of functional group selected from the group consisting of alkyl group having a carbon number 1 to 3.

The compound shown in the formula (I) is preferably that sown in the formula (II), because it has high promoting activities of bone density, growth of bones, formation of the cortical bone or cancellous bone.

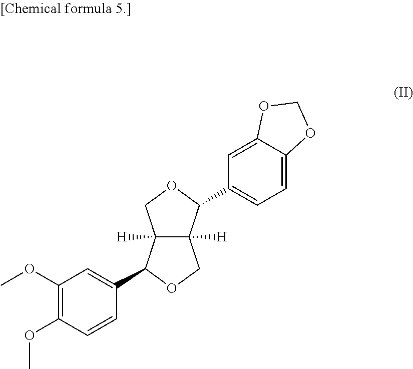

Wherein, the derivative includes the physiologically acceptable salt thereof, hydrate thereof, and a glycoside thereof, and a mixture thereof. As the physiologically acceptable salt thereof, there are mentioned such as sodium salt, potassium salt, chloride salt and the like. Also, as the physiologically acceptable hydrate, there are mentioned such as monohydrate, dehydrate and the like.

The compound shown in the formulae (I) to (III) and analogs thereof, salt thereof, hydrate thereof, glycoside thereof, and mixture thereof may be prepared by using the conventional method or according to that to obtain. Commercially available ones may be purchased.

For example, the composition of the present composition may be produced as described below.

Firstly, an organ selected from the group consisting of the flower bud, leaves, bark, and xylem is collected to dry to obtain dried ones.

Concretely, for example, Boushunka, dried flower bud of Magnolia biondii is prepared. The dried flower bud may be prepared to collect them from such plants by using air drying. The commercially available one sold as Shinni as the crude drug may be purchased to use. Instead of the flower but, the leaves, the bark or the xylem may be used.

The dried one is weighted at a predetermined weight; then about 1.7 to 7 times volume of the weight of methanol is added to perform the extraction at the predetermined temperature. Solid content is separated by filtration from the extract. Then, methanol is removed and the weight of a residue is weighed. Two to five volumes of the residue weight of the mixture of water/ethyl acetate is added to the residue to perform a partitioned extraction at the predetermined temperature.

When 1 kg of the dried flower bud is used, about 1.7 to 7.0 L of hydrous or non-hydrous alcohol is added, for example, about 0.085 to 70 L of 100% methanol is added to 0.05 to 10 kg of the dried flower bud, to perform the extraction at 3 to 14 days at 2 to 6 degree centigrade.

From the obtained extract, the solid content is separated by using a device such as Buchner funnel and the like. Subsequently, solvent is removed by using a rotary evaporator, flush evaporator and the like.

The amount of the residue is weighed, and then, for example, about 2 to 5 time volume of the mixture of water/organic solvent is added to be transferred into a separatory funnel. After that, it is subjected to the partitioned extraction at room temperature.

For the partitioned extraction, in addition to the partitioned funnel, a liquid-liquid extraction equipment, counter-current extraction equipment, and the like may be chosen, depending on the volume of the dried flower bud or other dried one. Also, for the partitioned extraction, water/ethyl acetate, water/acetone, water/butanol, and the like may be used. Among them, water/ethyl acetate is preferably used, because it is easier to remove the solvent from the organic phase. Particularly, it is preferable to set the ratio of water/ethyl acetate to 0.5/2 to 2/0.5 from the view point of extraction efficiency, more preferably 1/1.

After the partitioned extraction, the organic phase is separated from the aqueous phase. Then, the organic solvent of the organic phase obtained is removed by using the evaporator and the like to obtain the first concentrated solution. When the many components are extracted in the organic phase, the aqueous phase is separated from the organic phase. Then, the same organic phase to be separated is added to the organic phase at the same volume, and then separated. The procedure is repeated. By this, the intended compounds are extracted more, and the intended compound is obtained at high efficiency.

Then, the first concentrated solution is performed to the second partitioned extraction by using the different solvent system. Particularly, the mixture of the organic solvent is added to the first concentrated solution at 2 to 5 times volume of the first concentrated solution to perform the second partitioned extraction at the predetermined temperature. In order to eliminate aliphatic components, the solvent system such as n-hexane/water, n-hexane/methanol and the like is preferably employed. When n-hexane/methanol is employed; aqueous methanol including about 10% of water is preferably employed. If desired, the repeated extraction by using n-hexane has an advantageous effect that it accelerates elimination of the aliphatic components, and makes purification hereinafter easier. After that, obtained MeOH phase is separated similarly that as mentioned above to concentrate to obtain the second concentrated solution.

Not that the composition for prophylaxis and/or treatment of the bone disease may be produced from the according to the conventional method by removing MeOH to obtain crystalline.

Next, according to the following procedure, 90% MeOH fraction was purified by using a column chromatography to obtain one of the compounds of interest, fargesin.

Firstly, for example, an open column of diameter from 5 to 20 cm×length 12.5 or 75 cm is prepared, and 200 to 800 g of silica gel is packed into it. The first solvent is poured into the column to swell the silica gel. After the swelling of the gel, the second concentrated solution is applied on the gel to be fractionated by using a step gradient method to obtain the first fractions. The volume of the fractions may be properly decided, it is preferable to set the fraction as 0.75 to 1.5 L, because of the operability efficiency.

In the step gradient method employed here, for example, the elution solvent may be sequentially changed in stepwise ethyl acetate: n-hexane=1:9 to 10:0. Finally, for elution of the components adsorbed on the silica gel, 100% MeOH is employed. The content of the intended component may be confirmed by using a thin layer chromatography.

The mixture ratio of the elution solvent to obtain the intended compound of the composition of the present invention is preferably ethylacetate: n-hexane=1:9 to 7:3, more preferably 2:8 to 5:5, far more preferably 3:7. The mixture ratio of the elution solvent to have the highest yield is changed depending on the volume of dried flower bud, quality, solvent volume used for the subsequent extraction operation, the extraction temperature. Therefore, it is preferable to confirm the yield of each fraction by using the thin layer chromatography.

Note that the crystalline may be sometimes precipitated in the fraction including high content of the intended compound. In this case, the precipitates are separated by the filtration to be recrystallized according to the conventional method to obtain the crystalline with high purity.

Other fractions are processed as the same as the above-mentioned concentrated solution to obtain the pharmaceutical preparation for osteogenesis promotion of the present invention. The first fractions of them are concentrated as the same as those described above, they are purified the following procedure by using the preparative chromatography.

For example, the concentrated solution of the first fractions is applied onto a reverse phase column chromatography by using the octadecyl silica column (C₁₈-ODS), the inner diameter 2 cm×the length 20 cm; then fractionated by using the preparative chromatography. As the elution solvent, for example, water/methanol, of which mixture ratio are changed by 20%, may be employed. In this case, as the same as the case in which the open column is used, the purification may be performed by using the step gradient method.

The fractions are collected to the same ratio, and they are concentrated, checking the intended compound content. When the intended compounds are included in the 80% methanol fraction to give the precipitate as the crystalline by the concentration, the crystalline may be obtained by filtration of the concentrated fraction with, for example, No. 2 grade filter paper.

The obtained crystalline is dissolved in the predetermined solvent, and they are subjected to mass spectrometry (MS), nuclear magnetic resonance spectroscopy (NMR). Then, the obtained spectrum data are compared to the reference data to identify the structure of the obtained compound.

By using thus obtained compound or the composition (partially purified fractions), the pharmaceutical preparation for osteogenesis promotion, the functional food and the health food may be produced.

Note that the optimal dose of the compounds or the compositions for the human administration is generally about 50 times of that for the mouse. In one example, when it is administrated to the body weight 20 g of the mouse as the dosage of 20 mg/kg body weight/day, or to the body weight 35 g of the mouse at the dosage form of 100 mg/kg body weight/day are respectively converted to the dosage of 20 mg/day or 175 mg/day in human.

The second aspect of the present invention is the pharmaceutical preparation for osteogenesis promotion comprising the above-mentioned compound as the active ingredient. As the pharmaceutical preparations, there are mentioned non-parenteral dosage from such as injections, suppositories, aerosols, percutaneous form and so forth, parenteral preparations such as tablets, powders, capsules, pills, trochiscus, solutions and so forth. Wherein, the above-mentioned tablet includes sugar coated tablets, coat tablets, and buccal tablets; the capsule includes both of hard capsules and soft capsules. The granules contain coated granules. The above-mentioned solution contains suspensions, emulsions, syrups, elixirs, and so forth, and the syrup includes also dry syrups.

Other pharmaceutical preparations include the liquid formulation of the above-mentioned compositions, or the gel formulation preparation which is an impregnated gel the liquid form and the like. Note that the above-mentioned formulations include both of the sustained and non-sustained release formulation.

These preparations may be formulated according to the known pharmaceutical method by using pharmacologically acceptable carrier, excipient, disintegrator, lubricant, colorant, and so forth, for formulating the preparation, described on Japanese Pharmacopoeia.

As these carriers or excipients, for example, there are mentioned such as lactose, glucose, sucrose, mannitol, potato starch, corn starch, calcium carbonate, calcium phosphate, calcium sulfate, crystalline cellulose, powdered glycyrrhiza extract, powdered gentian, and so forth.

As the disintegrator, for example, there are mentioned such as starch, agar, powdered gelatin, sodium carboxymethylcellulose, calcium carboxymethylcellulose, crystalline cellulose, calcium carbonate, sodium bicarbonate, sodium alginate and so forth; as the lubricant, for example, there are mentioned such as magnesium stearate, talc, hydrogenated vegetable oil, macrogol and so forth.

The colorant, which is acceptable to be added to the pharmaceutical preparation, can be used with no limitation. Except these additives, a corrigent and so forth cam be used depending on the necessity.

When formulating the tablet or the granule, if necessary, they may be coated by using sucrose, gelatin, hydroxypropylcellulose, purified shellac, gelatin, glycerin, sorbitol, ethylcellulose, hydroxy-propylcellulose, hydroxypropylmethylcellulose, polyvinylpyrrolidone, acetate cellulose phthalate, hydroxypropylmethylcellulose phthalate, methylmethacrylate, methacrylate polymer, and so forth to have single coating or plural coatings.

Furthermore, the capsule can be prepared by encapsulating the granule or powdered preparation into the capsule made of ethylcellulose, gelatin, and so forth.

When the injection is prepared by using the above-mentioned compound, the physiologically acceptable salt thereof, or the hydrate thereof, a PH regulator, a buffering agent, a stabilizer, a solubilizing agent, and so forth may be added as needed.

When the preparation for preventing and/or treating bone disease is administrated to a patient, the dosage is depending on conditions such as thickness of the symptom, age, weight, and health status and so forth. In general, the preparation is administrated for an adult in the parenteral or non-parenteral route, at the dosage of 1 mg/kg to 2,000 mg/kg, preferably 1 mg/kg to 1,000 mg/kg once a day or more. In the present invention, it is preferably administrated at the dosage of 10 to 350 mg/day per an adult, and more preferably 20 to 175 mg/day per the adult. Number of administration and amounts a day may be adjusted depending on the conditions described above optionally.

When the content of the compounds as the active ingredient shown in the above-mentioned formula (I) is less than the lower limit, it does not show sufficient osteogenesis effects. In contrast, when the amount excesses the upper limit, it does not show the effect corresponding to the added amount. (Alternatively, it may cause potential undesirable side effect to a living body being administrated it.)

Here, in order to form the powdered composition of the present invention, the extract obtained in the production process may be condensed, and dried by using the method such as lyophilization, spray-drying, vacuum-drying and so forth; and then dried extract is pulverized into fine powder. Corn starch, dextrin, cyclodextrin, oyster shell powder may be added as needed

Alternatively, the above-mentioned binder is optionally added to the powder obtained as described above and compressed to from the tablet. After formulation of the tablet, it may be coated by using the coating agent such as sucrose, gelatin and so forth to formulate the sugar-coated tablet, or coated by other coating agent to formulate enteric coated tablet.

Furthermore, the powder obtained as describe above may be granulated by using the conventional method to formulate the granule. The powder or granule as mentioned above is encapsulated into capsules in a proper amount to formulate the capsule.

The above-mentioned compositions are added to, for example, breads, cookies, biscuits, wheat to be supplemented to rice and cereals, noodles such as Japanese wheat noodles, buckwheat noodles, pasta and others, dairy food such as cheese, yogurt and others, jam, mayonnaise, soy bean product such as soy bean paste, soy source and others, nonalcoholic beverage such as tea, coffee and cocoa, soft drinks such as and fruits juice, alcoholic beverage such as medicated liquor, snacks such as candy (drops), and chocolate, chewing gum, Japanese cracker, azuki-bean jelly and so forth, to produce the functional food or healthy food having preventing or treatment effect of periodontal disease.

Note that the compositions is added to yogurt, soy source, drinks and the like, solubilizing auxiliaries or the stabilizers may be employed not so as to form crystalline of the composition of the present invention to precipitated.

The composition of the present invention may be employed solely, or as a combination of 2 or more to formulate the powders, the granules, the tablets or the capsules to produce the healthy food.

The food taken by the bone disease patients for the predetermined period, in predetermined number and amounts promotes the cancellous bone formation of them to effectively prevent the appearance of the bone disease such as osteoporosis and the like.

The present invention is explained in detail by using examples below, however, the present invention is not limited to them.

### Example 1

### Study on the Change of Bone Mass in Type 1 Osteoporosis Model Animal

### (1) Test Animal

Female Slc: ddy mice of 4 weeks old (Japan SLC, Inc.) was operated with ovariectomy to use as a type I osteoporosis model animal. Also, in order to delete the affection by invasion, a group which is operated without ovariectomy (Sham operation) is set (Sham group). These were the aged model animals.

They are maintained under the condition of 12 hour light/dark cycle, at temperature of 23±3 degree centigrade, and humidity of 55±5% moisture in TP-102 (Toyo-Rico Co., Ltd.) cages at 4 mice/cage. Animal bedding were changed twice a week and always to use all fresh one, CRF-1 (Oriental Yeast Co., Ltd.) is fed as feed and deionized water as drinking water were freely given. Their body weights were weighed twice a week by the end of the experiment.

### (2) Preparation of Test Substance

(2-1) Partial Purification of Compounds Derived from Shini

Thirty five L of methanol is added to 10 kg of Shini (Magnolia kobus (M. praecocissima)) provided in Shan Xi Province in China to immerse to perform extraction at 4 degree centigrade for 7 days. A solid substance was separated by the filtration to obtain filtrate. Then, the whole amount of filtrate was concentrated by using the evaporator to obtain a crud extract.

Next, the crude extract was poured into 5 L volume of the separatory funnel, and 2 L of water/ethyl acetate (1/1) was added. The funnel was shaken to perform the partitioned extraction. The obtained ethyl acetate phase was concentrated by using the evaporator to be poured into another 5 L of the separatory funnel. Two L of hydrous methanol (10% of water content ratio)/hexane was added into the funnel to perform the partitioned extract again.

The obtained aqueous methanol phase was concentrated by using the evaporator, and then it was subjected to column chromatography under the following conditions. Step gradient method was employed for elution, by using the following elution buffer to obtain fractions corresponding to each elution buffer (one fraction=2,000 mL). The obtained fractions corresponding to the 30% ethyl acetate and 50% ethyl acetate were used as partially purified Magnolia kobus.

Column: an opened column with a diameter of 9 cm×a length of 50 cm, filled with a 400 g of Silica gel (BW-820 MH, Fuji Silica Chemical Co.).
Elution buffers: ethyl acetate/hexane (10/90, 20/80, 30/70, 50/50, and 70/30). Since precipitates (crystalline) were formed in the fractions of ethyl acetate/hexane=30/70 and 50/50, they were filtered. The obtained precipitates were 24.5 g.

### (2-2) Analysis of the Precipitated Crystalline

The precipitates obtained as mentioned above were analyzed by using LC-NMR according to the known method. HPLC conditions were shown in below. Its spectrum of ¹H NMR and ¹³C NMR was measured to determine its chemical structure. Optical rotation was measured to perform their structural analysis.

HPLC instrument: LC-8020 (Tosoh Corporation)

### Detector: UV-8011

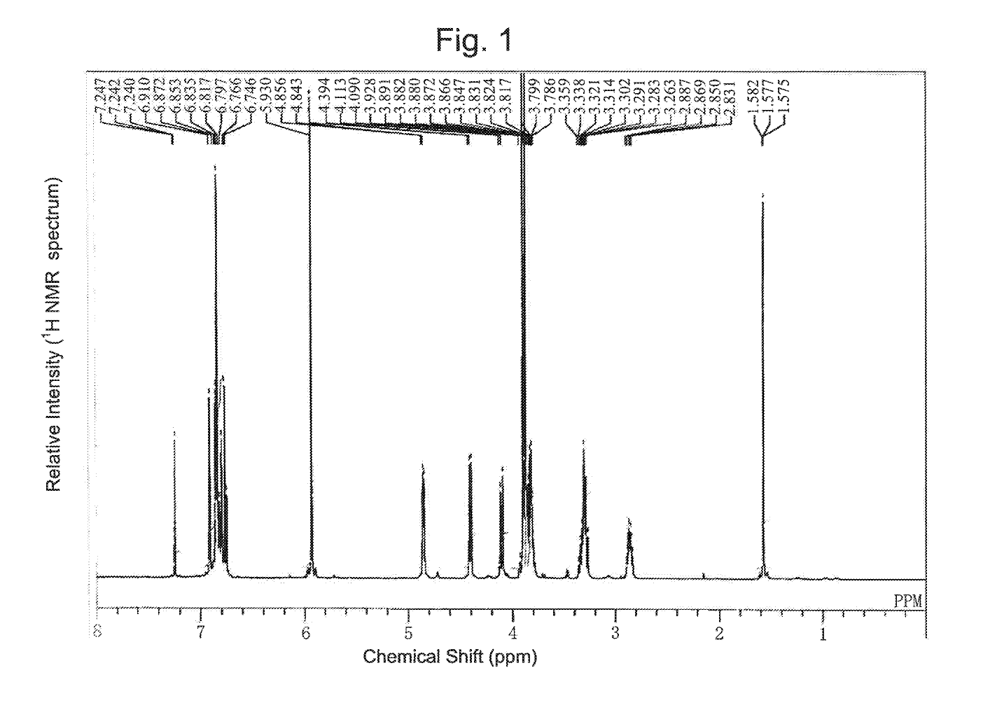

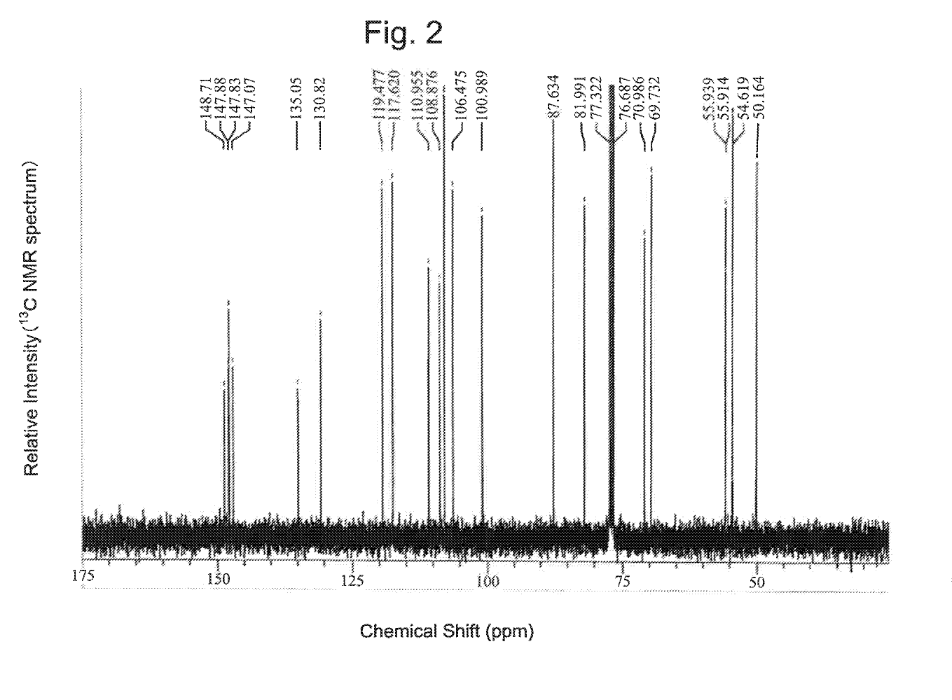

Column: Cholester waters φ4.6×250 nm (Nacalai Tesque)
Dissolution Medium: 50% acetonitrile/50% water
Solute temperature: room temperature
Solute concentration: 1 mg/ml
Injection volume: 2 μL
Flow rate: 1 mL/minute
Detective wavelength: UV 215 nm
NMR conditions were as follows:
NMR instrument: JNM-AL-400: FT NMR (400 MHz, JEOL Ltd.)
Solvent: Deuterated chloroform (CDCL₃)
Results of the ¹H NMR spectrum and the ¹³C NMR spectrum are shown in FIG. 1 and FIG. 2, respectively. Since the NMR spectrum data and the optical rotation data were completely correspond to those of fargesin (non-patent document 2), the obtained compound was identified as fargesin. The purity was 98%.

### (3) Test Method—Administration of a Test Substance

### (3-1) Preparation of the Test Substance

Fargesin was dissolved in 30 mL of a solution (it is referred to as “TD solution”, hereinbelow) containing 4% dimethyl sulfoxide (it is referred to as “DMSO”, hereinbelow) and 4% TWEEN 80 (both were purchased from Wako Pure Chemical Industries, Ltd.) so as that a dosage amount of fargesin becomes 20 mg/kg body wt/day, or 10 mg/kg body wt/day.

β-estradiol 4% dimethyl sulfoxide (refer to DMSO thereafter) was dissolved in 30 mL of an aqueous solution containing 2% DMSO to prepare β-estradiol solution so as that the dose amount of it becomes 100 μg/kg body wt/day.

### (3-2) Administration

After the surgical operation, the mice were adapted to the circumstances for 5 days, and then they were divided to the Sham group, the negative control, the positive control group, and the test substance administration group (6 mice per group). The ovariectomized mice were used for the negative control group, the positive control group, and the test substance administration group.

The aqueous solution including 4% DMSO was daily administrated to the mice with Sham operation group and the negative control group (they were collectively referred to as “NC group”) p.o. for 3 months. β-estradiol solution was daily administrated to the ovariectomized mice at the dose of 100 μg/kg body wt/day (it is referred to as “B100 group” thereafter) i.p.

Fargesin solution was daily administrated to the ovariectomized mice in the test group at the dosage of 20 mg/kg body wt/day (it is referred to as “F20 group”, hereinbelow) or 100 mg/kg body wt/day (it is referred to as “F100 group”, hereinbelow) for 3 month p.o.

### (4) Study on Effects to Bones

### (4-1) Preparation of the Bone Sample

In order to study effects of fargesin on the bone, the femur, the largest long bone, was used.

The mice in each group were killed by the cervical dislocation under diethyl ether anesthesia. Their right femurs were excised with muscles, and the muscles were removed from the femur after the excision. Lengths of the excised bones from the mice in the groups were measured, and they were dipped in 70% ethanol to be fixed.

### (4-2) Measurement of Bone Density and the Like

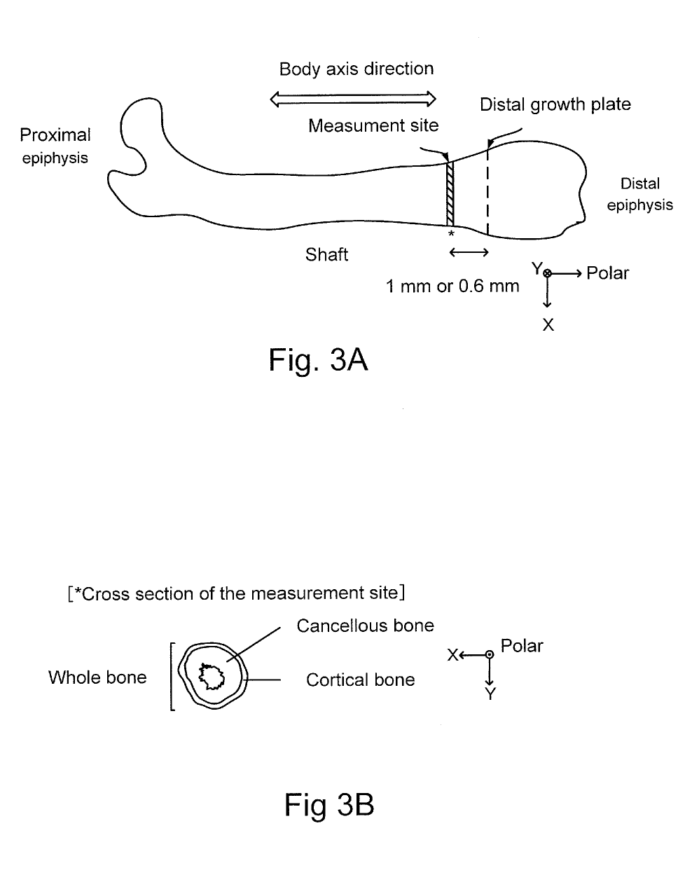

In FIG. 3, the bone was shown schematically. The long bone (tubular bone) was composed of two thick and rounded ends (epiphysis), and thin and long part between them (a shaft). The bone extends to longitudinal direction, depending on the growth of a plate-shape epiphyseal cartilage (a growth plate) between the epiphysis and the shaft. The region of 1 mm proximal side from the A position for a measurement was set to that from 1 mm proximal region (epiphysis) from the distal growth plate as decided as the measurement region.

The bone density was measured by using the peripheral quantitative computed tomography (pQCT, XCT Research SA+, Stratec Medizintechnik GmbH, Merk Ltd) (FIG. 3B). The measurement was performed under the conditions: a diameter of 90 mm, a voxel size of 0.12 mm, a CT speed of 10 mm/sec, and a block number of 1. Bone mineral content (mg/mm), bone density (mg/cm³), and cross section of the bone (mm²) of the entire of tomographic images (whole bone area) were calculated.

Here, the term “bone mass” means the sum of the bone mineral density and amount of protein substrate. The value of the bone mineral amount divided by the section of the bone is bone mineral amount per unit volume (the bone density).

Next, the area of the cancellous bone was extracted (peel mode 20), and then the bone mineral amount (mg/mm), the section of the bone (mm²), and the bone density (mg/cm³) were calculated. Further to the cortical bone, the bone mineral amount (mg/mm), the bone density (mg/cm³), the section of the bone (mm²), a bone thickness (mm), the periosteum perimeter of the cortical bone (mm), and the endosteum perimeter of the cortical bone (mm) were calculated.

### (4-3) Measurement of Bone Strength

Based on the bone diameter and the bone density measured by the pQCT, SSI was calculated. SSI is composed of Polar SSI (torsion strength in polar coordination), X-axis SSI (strength in X axis, three-point bending strength), and Y-axis SSI (Y axis strength, three-point lateral bending strength), and it is expressed by the following general equation.

SSI=Z×CBD/ND

Wherein, Z is the coefficient of the cross section (mm³), CBD is the bone density of the cortical bone (mg/cm³), and ND is the physiological bone density (1200 mg/cm³). The coefficient Z is shown as the following equation.

Z=(rₒᵤₜₑᵣ⁴−rᵢₙₙₑᵣ⁴)/rₒᵤₜₑᵣ×π/4

rₒᵤₜₑᵣ; outer diameter (mm), rᵢₙₙₑᵣ; inner diameter (mm)

The measurement sites were as mentioned above. A direction from the proximal epiphysis to the distal epiphysis is defined as the polar direction, X axis direction is defined as a horizontal direction away from the body axis, and Y axis direction is defined as vertically downward direction (FIGS. 3A and B). The polar coordinate torsion strength, X axis strength, and Y axis strength were respectively calculated. For the analysis of the obtained measurement data, Makejob (Stratec Medizintechnik GmbH., Germany) was used.

### (5) Test Results

### (5-1) Change of Body Weight

Average weights of the mice in each group were 19.8 g to 21.2 g at 4-week-old, 29.2 to 31.5 g at 8-week-old. At 12 week-old, the mice became almost mature and their weights reached 32.1 to 36.4 g. Average weight of B100 group mice was lower than that of NC group mice during mouse growth. However, the average weights of F20 group mice and F100 group mice were equal to that of NC group mice during the mouse growth.

### (5-2) Effect to the Whole Bones

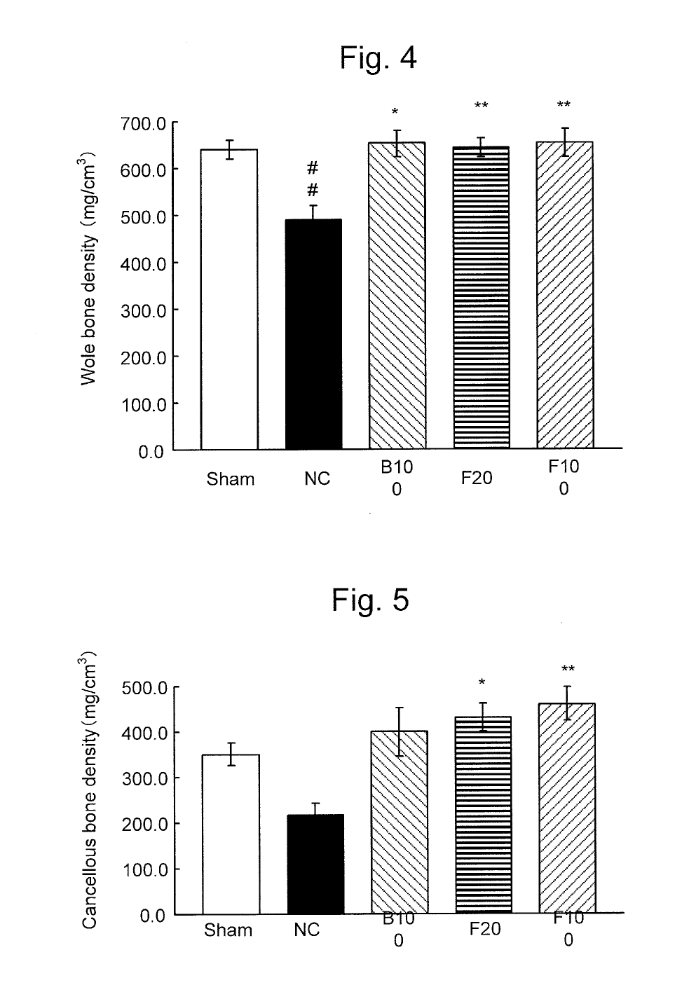

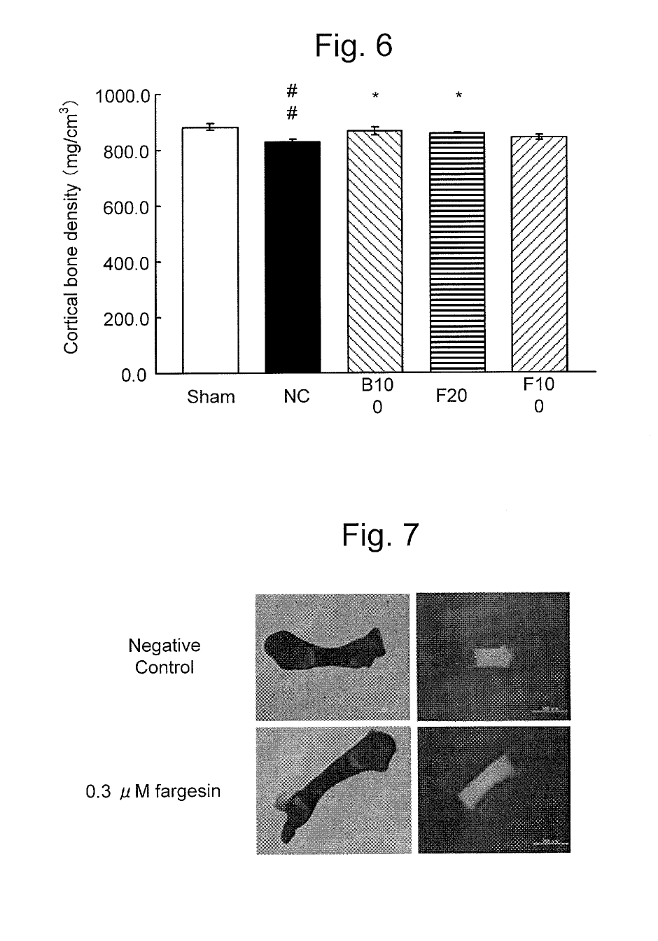

The bones were classified into the whole bone, the cancellous bone, and the cortical bone (a compact bone), the change of the bone mineral mass, the cross-section area, and the bone density were studied. Results are shown in Tables 1 and 2, and FIGS. 4 and 5. In Tables, the number written in lower side in each column shows relative value, when the data of Sham group is 100. The data was statistically processed by using Dennett's two-sided t test to decide whether there is significant difference or not. Each number in the table was shown in average ± standard error. From Tables 3 and after that were the same as Tables 1 and 2.

In the whole bone, the bone density of NC group was significantly lower than that of Sham group. Also, the bone mineral mass of NC group (2.252±0.239 mg/mm) was decreased compared to that of Sham group (3.270±0.234 mg/mm). In the cross sections of both bones, there was no significant difference between these groups. However, the cross section of the bone of Sham group reduced approximately 10% from 5.100±0.266 mm² to 4.558±0.169 mm². Thus, it was show that the ovariectomized mouse can be a model animal of Type I osteoporosis.

On the other hand, the bone mineral mass and bone density were significantly increased in the B100 group compared to those in the Sham group (bone mineral mass 3.325±0.203 mg/mm) to show preventative effects of osteoporosis (Table 1, FIGS. 4 and 5).

In the F20 group, not only the bone mineral mass (3.638±0.164 mg/mm) and the bone density but also the cross section area of the bone (5.643±0.152 mm²) were significantly increased. In the F100 group, not only the bone mineral mass (3.660±0.326 mg/mm) and the bone density but also the bone cross section (5.550±0.351 mm²) were similarly significantly increased to show that bone growth was promoted.

TABLE 1

| Bone density (mg/cm³) | | whole bone | Cancellous bone |
|---|---|---|---|
| | Sham | 639.450 ± 20.653 (100) | 350.750 ± 24.892 (100) |
| Negative control | NC | 488.460 ± 32.809^{##} (76.4) | 216.080 ± 26.302 (61.6) |
| group | | | |
| Positive control | B100 | 652.025 ± 28.386* (102.0) | 399.050 ± 53.851 (113.8) |
| group | | | |
| Test group | F20 | 644.367 ± 20.387** (100.8) | 431.283 ± 30.506* (123.0) |
| | F100 | 653.300 ± 29.083** (102.2) | 460.500 ± 36.222** (131.3) |

^{#}p < 0.05 against Sham,
^{##}p < 0.005 against Sham
*p < 0.05 against NC,
**p < 0.005 against NC

Next, the effects of β-estradiol and fargesin on the bone at following regions were studied.

### (5-3) Effects on the Cancellous Bone Region

Compared to Sham group (the bone mineral mass 0.6333±0.077 mg/mm, the cross section areas of the bone 1.787±0.091 mm², the bone density 350.750±24.892 mg/mm³), those of NC group were as follows: the bone mineral mass was 0.354±0.091 mm², the cross sectional area of the bone was 1.606±0.058 mm², and the bone density was 216.080±24.892 mg/mm³, and the trends were the same as those in the whole bone B100 group whose bone mineral mass is 0.725±0.110 mg/mm and the bone density was 399.050±53.851 mg/cm³, and they showed increased trend, but they did not show any significant difference between them.

In contrast, in F20 group, the bone mineral mass was 0.855±0.067 mg/mm, and the bone density was 431.283±30.506 mg/cm³. In the F100 group, the bone mineral mass was 0.920±0.102 mg/mm and the bone density was 460.500±36.222 mg/cm³. Compared to those of the NC group, the bone mineral mass and the cross section area of both groups were significantly increased (Table 2, FIGS. 4 and 5). Therefore, it was demonstrated that fargesin maintained the bone mineral mass of the cancellous bone, which would be decreased by the ovariectomization, and increase the bone density.

### (5-4) Effects on Cortical Bone Region

In the Sham group, the bone mineral mass was 2.192±0.180 mg/mm, the cross section area was 2.477±0.182 mm², the bone density was 822.083±12.665 mg/cm³, and the bone thickness was 0.361±0.023 mg/mm. In contrast, the bone mineral mass was 1.150±0.246 mg/mm, the cross section area of the bone was 1.390±0.277 mm², the bone density was 826.540±11.518 mg/cm³ and the bone thickness was 0.206±0.040 mg/mm in the NC group. In the NC group, since all of the values were significantly decreased. Particularly, the bone mineral mass, the cross section of the bone, and the bone thickness were severely decreased, it was demonstrated that the cortical bone became thinner and brittler.

In contrast, the bone mineral mass was 2.163±0.230 mg/mm, the cross section area of the bone was 2.4825±0.230 mm², the bone density was 867.475±13.724 mg/cm³, and the bone thickness was 0.365±0.038 mg/mm in the F100 group. Compared to those of the NC group, since all of the values were significantly increased, it was demonstrated that β-estradiol had highly effective for preventing brittle cortical bones.

On the other hand, the bone mineral mass was 2.087±0.160 mg/mm, the cross section area of the bone was 2.427±0.185 mm², the bone density was 859.483±2.479 mg/cm³, the bone thickness was 0.329±0.026 mg/mm in the F20 group. Compared to those of the NC group, the bone mineral mass, the bone density, and the cross section area of the bone were significantly increased. Also, in the F100 group, the bone mineral mass was 2.240±0.320 mg/mm, the cross section area of the bone was 2.640±0.361 mm², the bone density was 843.55±8.714 mg/cm³, the bone thickness was 0.370±0.051 mg/mm. Compared to those of the NC group, the bone mineral mass, the bone density, and the cross section area of the bone were significantly increased.

As described above, it was demonstrated that fargesin had effect for preventing the brittle cortical bone at the same level as that of β-estradiol. Further, it was demonstrated that there were a tendency for fargesin to have was highly effects than those of β-estradiol for boundary lengths of the bone adventia and bone endosteum (Table 2).

TABLE 2

| | | | | | Cortical bone |
|---|---|---|---|---|---|
| | | | Bone | Cortical bone | lining |
| | | Bone density | thickness | adventitia | membrane |
| Cortical bone | | (mg/cm³) | (mm) | diameter (mm) | diameter (mm) |
| | Sham | 882.083 ± 12.665 | 0.361 ± 0.023 | 7.9930 ± 0.207 | 5.727 ± 0.181 |
| | | (100) | (100) | (100) | (100) |
| Negative | NC | 826.540 ± 11.518^{##} | 0.206 ± 0.040^{#} | 7.5608 ± 0.139 | 6.301 ± 0.119 |
| control group | | (93.7) | (57.1) | (94.6) | (110.0) |
| Positive | B100 | 867.475 ± 13.724* | 0.365 ± 0.038* | 7.9895 ± 0.096 | 5.699 ± 0.229 |
| control group | | (98.3) | (101.1) | (100) | (99.5) |
| Test group | F20 | 859.483 ± 2.479* | 0.329 ± 0.026 | 8.4160 ± 0.116* | 6.347 ± 0.160 |
| | | (97.4) | (91.1) | (105.3) | (110.8) |
| | F100 | 843.55 ± 8.714 | 0.370 ± 0.051* | 8.3300 ± 0.278* | 6.010 ± 0.313 |
| | | (95.6) | (102.5) | (104.2) | (104.9) |

^{#}p < 0.05 against Sham,
^{##}p < 0.005 against Sham
*p < 0.05 against NC

### (5-5) Bone Strength

Polar axis strength (polar coordinates strength) was 1.599±0.143 mm³ in the Sham group, and 1.145±0.129 mm³ in the NC group. This showed that the bone strength index decreased in the NC group. On the other hand, it was 1.376±0.088 mm³ in the B100 group. There was the tendency for the NC group to decrease the strength; however, there was no significant difference between those groups.

In contrast, it was 1.615±0.090 mm³ in the F20 group, and was 1.666±0.087 mm³ in the F100 group. In those groups, the bone strength indexes were significantly increased as compared to NC group (Table 3).

Accordingly, it was demonstrated that fargesin largely increases the bone strength index than β-estradiol does. This shows that fargesin effectively prevents the brittle bone caused by the ovariectomy.

TABLE 3

| | | Polar axis bone strength |
|---|---|---|
| | | (mm³) |
| Negative control group | Sham | 1.265 ± 0.147 (71.6)^{#} |
| | NC | 1.601 ± 0.107 (86.1) |
| Positive control group | B100 | 1.946 ± 0.107 (101.0)* |
| Test group | F20 | 2.005 ± 0.154 (104.2)** |
| | F100 | 1.265 ± 0.147 (71.6)^{#} |

^{#}p < 0.05 against Sham
*p < 0.05 against NC,
**p < 0.005 for NC

### Example 2

### Organ Culture Test

### (1) Organ Culture of the Bone

Eagle's minimum essential mediums (MEM, Life Technologies Japan Corporation (Invitrogen)) was used to prepare the organ culture medium by adding 1% of penicillin/streptomycin (Life Technologies Japan Corporation (GIBCO)), 0.25% of fetal bovine serum (Sigma-Aldrich Co. LLC.), 50 μg/ml of ascorbic acid (Wako Pure Chemical Industries, Ltd.), 1 mM of β-glycerophosphoric acid (Sigma-Aldrich Co. LLC.), and 1 μg/ml of calcein (Sigma-Aldrich Co. LLC.). As a test substance, 0.3 μM of fargesin, or as a control, 0.3% of DMSO (final cone., Wako Pure Chemical Industries, Ltd.) was added to the organ culture medium. Fargesin was used the same solution prepared in Example 1 containing 0.3% DMSO as the final concentration.

On 15 of pregnancy, mouse fetuses were took from pregnant female ICR mice (Japan SLC, Inc.) by Caesarean operation (E15.5) from the 15^{th} days of a pregnant ICR female mouse by Caesarean section. Left and right metatarsals of the fetus were excised to be placed into the organ culture medium. The organ culture was performed under the conditions of 5% CO₂ at 37 degree centigrade for 7 days to observe the effect on long axis direction.

### (2) Observation Methods and Test Results

The long axis of the bone grows depending on that of epiphyseal cartilage existing between an epiphysis and a shaft. When the growth of the epiphyseal cartilage stops, calcium salt resulted in deposition of the substances around the cartilage cells. Then, calcification was occurred from the occification center (ossification). The calcified region was fluorescently-stained by using calcein, and then observed under the microscope. The observed image was shown in FIG. 7.

It was observed that the metatarsal was extending tot the long axis direction in the mediums as the increased calcified area by using the fluorescence of the calcein. As compared to the negative control without fargesin (0.3% DMSO addition), the metatarsal extended longer vertically in the medium with 0.3 μM of fargesin. As a result, it was shown that 0.3 μM of fargesin was enough for promoting the bone extension in the tissue concentration.

### Example 3

### Effects on Differentiation of Osteoblast and Osteoclast in Co-Culture

### (1) Preparation of Test Cells

Four-week-old ddY male mice (Japan SLC, Inc.) were killed by the cervical dislocation, and then, the long bones of left and right lower extremities were excised with muscles. All of the muscles were removed from the bones, and the femurs and shinbones were obtained. The both ends of the obtained femurs and shinbones were scraped a little by a little. The cells in the bone morrow were pushed out into the following medium by using a 2.5 ml of syringe with a needle (22G×1¼; Thermo corporation). After that, contaminants were eliminated through a filter (70 μM Nylon Cell Strainer; Japan Becton, Dickinson and Company) to obtain more than 2×10⁸ cells of BMCs.

Osteoblast-like cells, UAMS-32 cells, were purchased from the Institution of Physical and Chemical Research (Japan).

### (2) Preparation of Mediums

In order to prepare the basic medium, 10.2 g of α-MEM (powder, Life Technologies Japan Corporation (GIBCO)) was dissolved in 1 L of purified water and 2.2 g/l (w/v) of sodium bicarbonate was added. Then, it was filtered through a sterilized filter of which pore diameter was 0.22 μm (Nihon Millipore K. K). Further, the basic mediums were supplemented with 10% (v/v) fetal bovine serum (FBS) (Sigma, heat-inactivated for 30 minutes at 56° C.).

In this example, 1 μM of PGE₂ and 10 nM of Vitamin D₃ (both from Wako Pure Chemical Industries, Ltd.) were added to the medium to prepare a co-culture medium. By using the co-culture medium, cell suspensions of BMCs and UAMS-32 obtained as mentioned above were prepared. For the test group, 2 to 80 μM of fargesin was added, and for the negative control group, DMSO was added so as to be 0.3% of the final concentration.

### (3) Culture

BMCs were plated at the concentration of 2×10⁶ cells/well, and UAMS-32 cells were plated at the concentration of 1×10⁵ cells/well in each well of the 96 well plates, respectively. Then, the cells were co-cultured in the co-culture medium under the conditions of 5% CO₂ and 37 degree centigrade for 5 days. The medium were changed on the 3^{rd} days from the culture start. After cells were fixed as described in below, alkaline phosphatase (ALP) activity was measured as the index of osteoblast differentiation; tartrate-resistant acid phosphatase activity was measured as the index of osteoclast differentiation.

### (4) Study of the Effects on the Osteoclast Differentiation

The cells were cultured under the above-mentioned conditions, 10% formalin aqueous solution was added into each well still standing for 10 minutes, and then ethanol was added still standing for further 1 minute to fix the cells. In order to measure TRAP activity, 10 mM sodium tartrate/50 mM citric acid buffer solution containing 1.36 mg of p-nitrophenyl sodium phosphate (Sigma-Aldrich Co. LLC.) (pH 4.6) was prepared as a substrate solution for TRAP. The substrate solution for TRAP was added to the well at the volume of 100 μl/well, and reacted for 15 to 20 minutes at a room temperature. The reaction solution was transferred into another 96 well plate, to which 100 μl/well of 0.1N NaOH was previously added to stop the reaction, and they were measured at absorbance of 405 nm.

50 mM sodium tartrate/0.1 M of sodium acetate buffer (pH 5.0) including 0.1 mg/ml naphthol AS-MX phosphate (sigma N-4875) and 0.6 mg/ml Fast red violet LB salt (both from Sigma-Aldrich Japan Co. LLC.) was prepared as TRAP staining solution. The TRAP staining solution was added to the wells to stain the cells in negative control group till they took on red color under room temperature. After that, the cells were washed with distilled water. The cells stained red and having not less than two nuclei was decided as multinuclear osteoclast, of which number was counted by using the microscope.

### (5) Osteoblast Differentiation Test

After the cells were cultured under the conditions, 100 μl/well of methanol cooled to −20 degree centigrade was added into the cells and stood for further 1 minute to fix the cells. ALP substrate solution containing 2.47 mg/ml of 4-nitrophenyl phosphoric acid disodium salt hexahydrate (Sigma-Aldrich Japan Co. LLC.), 2 mM of MgCl₂ and 0.1M of Tris-HCL (pH8.5) was prepared.

ALP substance solution was added with the volume of 100 μl/well, and then the reaction was performed at room temperature for 15 to 20 minutes. Next, the reaction solution was transferred into another 96 well plate being added 100 μl/well of 0.1N NaOH to stop the reaction. Then, the plate was measured the absorbance at 405 nm, and set the value as the index of ALP activity.

As ALP staining solution, 0.1 M Tris-HCl (pH8.5) containing 0.1 mg/ml Naphthol AS-MX phosphate, 0.02% (v/v) N,N-dimethyl formamide, 0.6 mg/ml of Fast blue BB salt (Sigma F-3378, Sigma-Aldrich Co. LLC.), and 2 mM of MgCl₂ was prepared.

After adding the ALP staining solution, the reaction was performed at room temperature, until the cells of the negative control group were colored blue-violet. Then, the cells were washed. The strength of the staining was determined by visual observation to determine the ALP activity.

### (6) Statistical Application

All data was statistically analyzed by using SPSS (registered trademark) Statistics 17.0+Amos 17.0 (S.P.S.S. Jan Inc.) and described by using Average ± Standard Error (SEM). As a test, Dunnett's two-sided t test was employed. Significance level was shown as comparisons between the test group and the negative control group as follows: **: p<0.005, *<0.05. The sample number was more than 3 in each group.

### (7) Results

### (7-1) ALP Activity and TRAP Activity

The absorbance data obtained as described above was shown as the ratio of the activities, when the negative control was 100%.

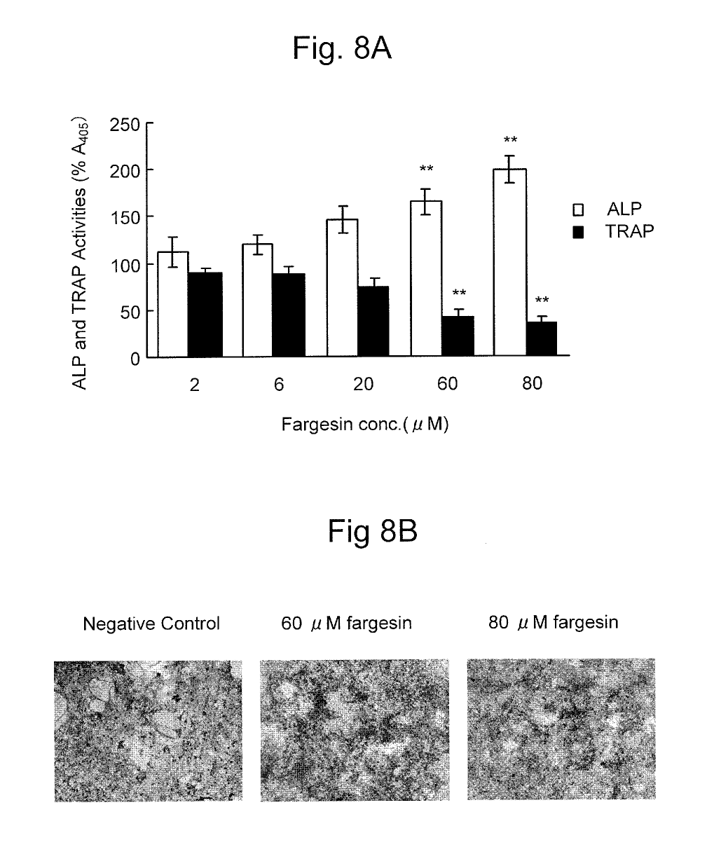

Against the negative control group, in the group with 2 to 80 μM fargesin, ALP activities were increased depending on fargesin concentration. However, TRAP activities were decreased in contrast. Particularly, in the group with 60 to 80 μM fargesin, the significant increase of the ALP activities and the significant decrease of the TRAP activities were shown (Table 4 and FIG. 8A). This shows that promotion of osteoblast differentiation and inhibition of the osteoclast differentiation were occurred.

TABLE 4

| Concentration | | |
|---|---|---|
| (μM) | ALP activity (%) | TRAP activity (%) |
| 0 | 100.0 | 100.0 |
| 2 | 112.6 ± 15.7 | 89.5 ± 5.4 |
| 6 | 119.8 ± 10.1 | 88.7 ± 7.2 |
| 20 | 145.6 ± 14.4 | 74.4 ± 8.5 |
| 60 | 164.3 ± 14.0** | 42.4 ± 7.1** |
| 80 | 198.9 ± 14.6** | 35.7 ± 5.6** |

**p < 0.005 for untreated cells

### (7-2) ALP and TRAP Double Staining

In the negative control group, both cells stained in red (the osteoclast) and in blue (the osteoclast) were observed. In contrast, in the group with 60 to 80 μM fargesin, the cells differentiated to the osteoclast were rarely observed (FIG. 8B).

From the observation results, it was demonstrated that fargesin activated the osteoblast; on the other hand, it inhibited the promotion of the differentiation from the osteoclast precursor cells to the osteoclast by the osteoblast. These facts show the excellent functions of fargesin, which effectively inhibit the increase of the bone resorption and reduction of the bone formation, and improve the balance of the bone reconstruction.

### Example 4

### Study of the Effects for Osteoblast Activation

(1) Test cells and culture conditions

MC3T3-E1 cells ((IAA) the Institution of Physical and Chemical Research), osteoblast-like cell line derived from the mouse fetus cranial bone cells, were inoculated into the 96 well plate at 4,000 cells/well. As a medium, the basal medium prepared in the Example 3 supplemented with fargesin at 2 to 80 μM was used. Pre-culture was performed by using the basal medium only under the conditions of 5% CO₂ and 37 degree centigrade for 3 days. After that, the conditioned medium was changed to fresh medium with fargesin; the culture was performed under the conditions of 5% CO₂ and 37 degree centigrade for 3 days. On the day 4 from the culture start, the conditioned medium was changed to the fresh medium; the culture was further performed under the conditions of 5% CO₂ and 37 degree centigrade for 3 days. After the termination of the culture, the following MTT test, measurement of ALP activities, and the staining were performed.

### (2) Measurement of Cell Viability

Cell viability was measured by using MTT test. MTT reagent was prepared by dissolving 50 mg of 3-(4,5-dimethyl-thiazol-2-yl)-2,5-diphenyl tetrazolium bromide in 10 ml of PBS (−). After the termination of the culture, the medium containing fargesin was partially removed from each well to adjust the solution volume to 100 μL. Ten μl of the MTT reagent was then added to each well to be reacted to give blue-violet color in the CO₂ incubator.

After the termination of the reaction, all medium was removed from each well, 100 μl of DMSO as a lysis solution was added to each well, and then the absorbance was measured at 570 nm to study the viability of live cells.

Measurement of the ALP activity, statistical processing of the data, and the observation of differentiated cells were performed as the same way as those in Example 3, except the staining of the osteoclast cells were omitted.

### (3) Test Results

ALP activity and the cell viability were shown in Table 8 as relative values when the negative control was 100.

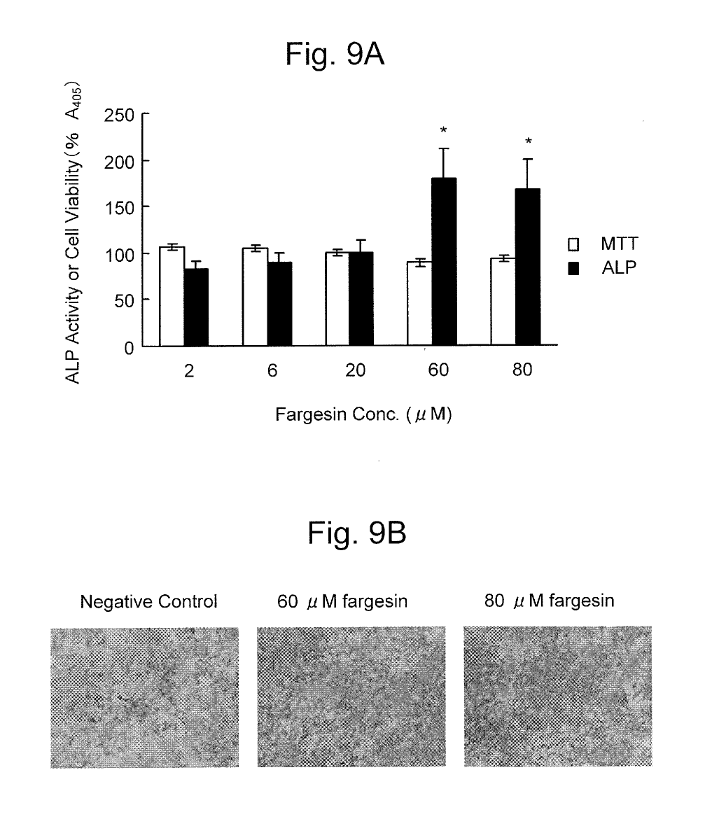

Against the negative control, the group with 20 to 80 μM fargesin did not show any significant difference in the cell viability (Table 5 and FIG. 9A). On the other hand, the group with 60 to 80 μM Fargesin showed a trend toward declining the cell viability, compared to the negative control group. In contrast, ALP activity was significantly increased (Table 5 and FIG. 9A). When 60 to 80 μM fargesin was added, many osteoblast stained in blue were observed (FIG. 9B).

TABLE 5

| Concentration (μM) | ALP activity (%) | MTT activity (%) |
|---|---|---|
| 0 | 100.0 | 100.0 |
| 2 | 82.8 ± 8.0 | 106.1 ± 3.2 |
| 6 | 89.8 ± 9.8 | 105.4 ± 3.3 |
| 20 | 99.8 ± 13.4 | 100.1 ± 3.4 |
| 60 | 179.8 ± 31.5* | 89.1 ± 3.9 |
| 80 | 167.3 ± 32.7* | 92.6 ± 3.7 |

*P < 0.005 against untreated cells.

From the observation results, it was clearly showed that fargesin activated the osteoblast-like cells even if the level did not any effects on their survival to promote the osteoblast cell function. This means that the fargesin did not cause any side effects and it was useful as the bone formation promotion agent.

### Example 5

### Study of the Effects on the Calcification of the Osteoblast

### (1) Test Cells and Culture Conditions

MC3T3-E1 cells as the same as those used in Example 4 were used. In addition, 50 μg/ml of L-ascorbic acid and 10 mM β-Glycerophosphate (both from Sigma-Aldrich Japan Co. LLC.) were added to the basal medium employed in the Example 3 was used. Fargesin prepared in the Example 1 was added to the medium the concentration at 2 to 80 μM.

MC3T3-E1 was inoculated in each well of the 96 well plates at 4,000 cells/well, and then the plate was pre-cultured by using only basal mediums under the condition of 5% CO₂ and 37 degree centigrade for 2 days. After that, the medium was exchanged to the medium containing 50 μg/ml of L-ascorbic acid, 10 mM β-Glycerophosphate, and fargesin, and it was incubated under the condition of 5% CO₂ and 37 degree centigrade for 5 days. After the termination of the incubation, the measurement of ALP activity and the differentiated cells were observed as the same as those in Example 3.

Mineral deposition was stained by using 1% alizarin red observed as the bone calcification (Wako Pure Chemical Industries, Ltd.) according to the conventional method, and then it was observed as the bone calcification.

### (2) Results

ALP activity was shown in Table 6 as relative values, when the negative control group was 100.

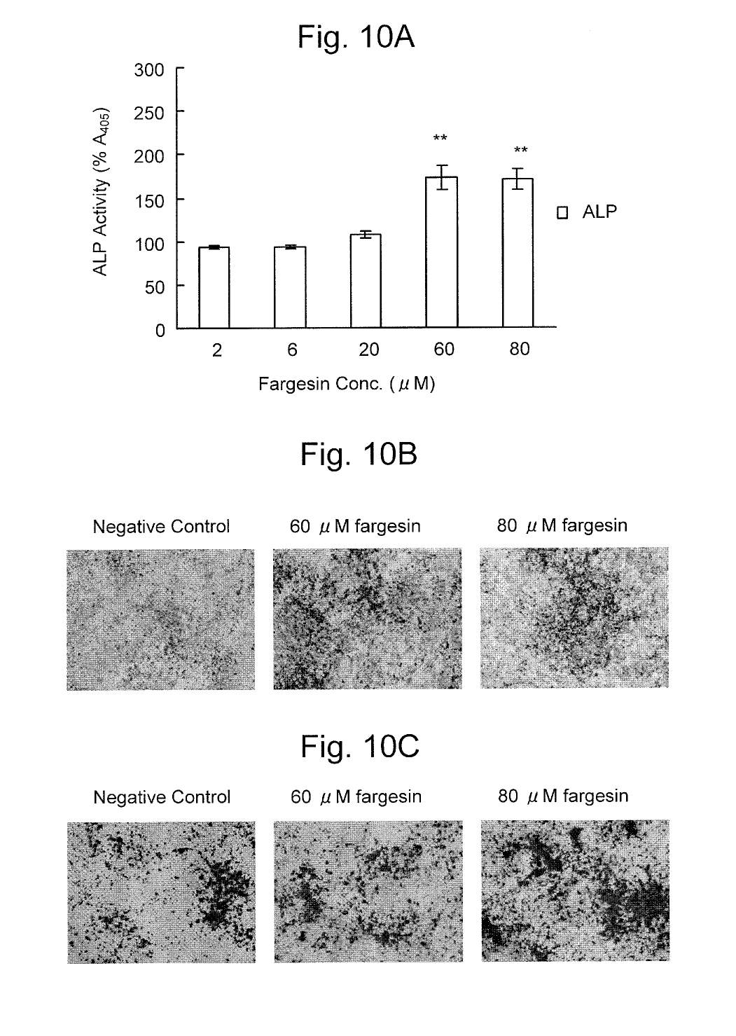

In the group supplemented with 60 to 80 μM fargesin, ALP activity was significantly increased, as compared to the negative control group (Table 6 and FIG. 10A). When 60 to 80 μM fargesin were added to the medium, many stained osteoclast cells were observed and ALP activity was higher (FIG. 10B). Further, when 60 to 80 μM fargesin was added to the medium, an increased mineral deposition side stained in red, namely the increased calcification site was also observed (FIG. 10C).

TABLE 2

| Concentration (μM) | ALP activity (%) |
|---|---|
| 0 | 100.0 |
| 2 | 93.3 ± 2.6 |
| 6 | 92.8 ± 1.6 |
| 20 | 107.0 ± 3.8 |
| 60 | 173.3 ± 13.7** |
| 80 | 171.5 ± 12.2** |

**p < 0.005 against untreated cells

From the results, it was obvious that fargesin promoted the osteoblast maturation and osteoblast calcification. This means that fargesin promotes the mechanical structure of the bone formation through the osteoblast activation. Also, the facts observed in the Example 3 to 5 showed that the effect for the living body of fargesin was supported by molecular mechanism in the cells.

### Example 6

### Evaluation of the Bone Formation of Fargesin by Using the Ovariectomized Mouse (OVX Mouse)

### (1) Test Animals

Four-week-old female Slc; ddy mice (Japan SLC, Inc.) were purchased, and they were divided into 8 groups without acclimate keeping (6 mice per group). They were anesthetized by using 50 mg/kg of pentobarbital to be ovariectomized (it is referred to as “OVX” herein below) or to have Sham operation (it is referred to as “Sham” herein below) under the anesthesia which is given by intraperitoneal administration of 50 mg/kg of Somnopentyl (pentobarbital sodium).

The reason why they are not kept was to prevent the increase of the weight of white adipose tissue, which causes mistake in treatment.

### (2) Test Methods—Administration of a Test Substance

From the second day of the ovariectomy (or Sham operation), each group was maintained under the condition of 12 hour light/dark cycle, 23±3 degree centigrade as the room temperature, and humidity 55±5% for 2 months as the bone resorption induction period. The animal bedding was exchanged twice a week and all fresh bedding was used at all times. CRF-1 (Oriental Yeast Co., Ltd.) as the feed and deionized water as drinking water were freely given.

After two month period from the test start, the bone resorption induction period, the test substance were given to each group at the amount shown in the following Table 7 for 3 month.

Ninety % of MeOH fraction (Shin-i) obtained in Example 1 and Fargesin (may refer to “Far” thereafter) were respectively dissolved into TD solution at the concentration shown in Table 7, and then they were administrated per os.

Human PTH (1-34) (herein below, it is sometimes referred to as “hPTH (1-34)”) were dissolved in distilled water at the concentration of 80 μg/kg/day, and it was administrated subcutaneously.

TABLE 7

| Name of the | Administered | | |
|---|---|---|---|
| group | solution | Route | Administration periods |
| Sham OVX | TD solvent only | p.o. | 3 months after the induction |
| | | | period passed |
| Control OVX | TD solvent only | p.o. | The same as above |
| hPTH(1-34) | 80 μg/kg/day | s.c. | The same as above |
| Shini | 90% MeOH fr. | p.o. | The same as above, Fargesin |
| | 40 mg/kg/day | | content corresponds to |
| | | | 2 mg/kg/ day administration |
| | | | group |
| Far 2 | 2 mg/kg/day | p.o. | The same as above |
| Far 20 | 20 mg/kg/day | p.o. | The same as above |
| Far 40 | 40 mg/kg/day | p.o. | The same as above |

### (3) Study of the Effect to the Bone

### (3-1) Preparation of the Bone Sample

After 3 month from the administration start, the mice belonging to all of the groups were weighed. They were anesthetized by using diethylether, and were collected their blood from their hearts, and then they were died. Then, organs such as the uterus, the white adipose tissue (WAT), the brown adipose tissue (BAT), the liver, the spleen, and both left and right lower extremities were removed. The left and right lower extremities were separated into the femurs and the tibias, and they were stored in 70% EtOH under the room temperature.

### (3-2) Measurement of the Bone Density, Tissue Weight and the Like

The muscles were removed from the obtained femur (right), and then its bone density and the like were measured by using pQCT employed in Example 1 under the following conditions.

<Measurement Conditions for pQCT>

Voxel size (mm): 0.07

Recognition of contour (CONTMODE): 2 (auto search of ROI)

PEELMODE: 20

Recognition method of cancellous bone: Surface & Area

Ration of cancellous bone area to all of the cross section areas: 35%

CORTMODE used in CORTBD: 1* * Voxels not larger than the standard value for distinguishing the cortical bone from the cancellous bone (threshold: threshold) were omitted. Each tissue weight was also measured.

TH value used in CORTBD: 690

### (3-3) Measurement of Serum TRACP5b and Osteocalcin Concentrations (Markers of the Bone Metabolism)

All obtained blood from the heart under diethyl ether anesthesia at dissection was stored at 4 degree centigrade for 24 hours. Then, the blood was centrifuged at 3,000 rpm (4,000×g) for 15 minutes to separate the sera.

According to the protocol of Mouse TRAP™ Assay (Immuno-diagnostic Systems Ltd, the United of Kingdom), TRACP5b in the obtained sera was determined.

Among the all data of cancellous bone density obtained from pQCT, both of the highest and the lowest data were deleted to obtain the Median. As to the other parameters were treated as the same as this, the graph was made with N=6. Statistical analysis was performed by using the Dennett's two-sided t test.

### (4) Test Results

### (4-1) Effects on the Body and Tissue Weights

The weights of the body, the uterus, BAT, WAT, the liver, and the spleens of each group were weighed. There were no biases depending on the groups. Also, since the weight gains of the uterus, the inhibition of atrophy, in both of PTH administration group and fargesin administration group were not shown, it was considered that these compounds did not have estrogen-like functions. The amount of feed intake (weight) of Sham group was slightly much than that of the OVX group. However, there was not much difference between them.

### (4-2) Effects on the Whole Bone and the Cancellous Bone

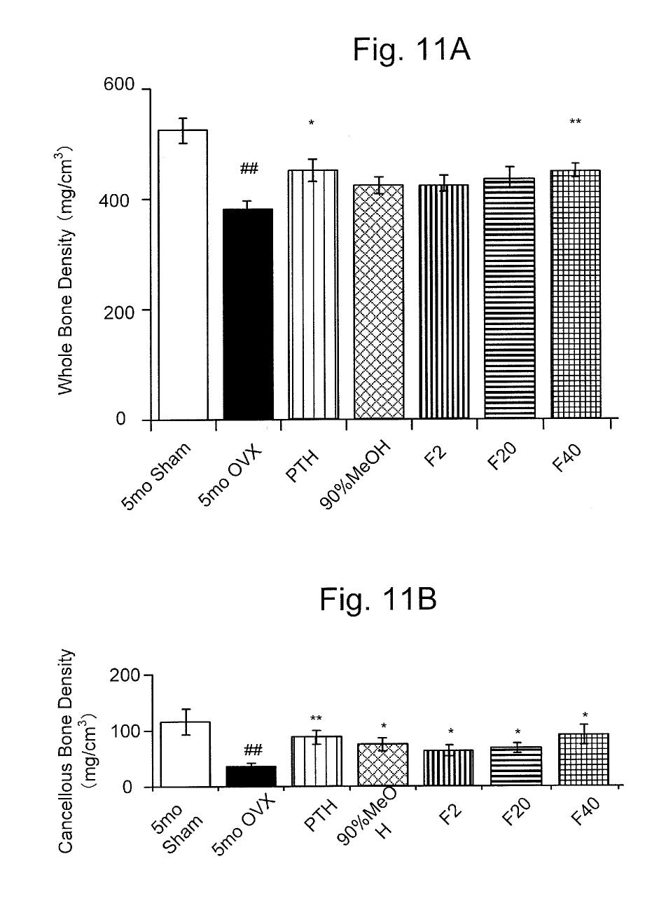

Whole bone mineral content, whole bone densities, and cortical bone mineral content were measured at the site of 1 cm away from the growth plate of the bone. The results were shown in Table 8, FIGS. 11A and 11B. At the time point of 5 months from the experiment start, the whole bone mineral content and the whole bone densities of the control OVX group were dominantly decreased as compared to those in Sham OVX group.

TABLE 8

| | Bone density of the | Bone density of | Bone density of |
|---|---|---|---|
| | whole bones | cancellous bone | cortical bone |
| | (mg/cm²) | (mg/cm²) | (mg/cm²) |
| 5M Sham | 525.65 ± 59.21 (100) | 116.65 ± 56.36 (100) | 983.95 ± 39.93 (100) |
| 5M OVX | 381.68 ± 37.65 (72.6)^{##} | 35.77 ± 16.96 (30.7)^{##} | 931.33 ± 37.78 (94.6) |
| hPTH(1-34) | 452.25 ± 49.00 (86.0)* | 88.83 ± 30.90 (76.1)** | 906.50 ± 32.19 (92.2) |
| 90% MeOH | 425.33 ± 38.64 (80.9) | 74.90 ± 29.38 (64.2)* | 888.52 ± 20.10 (90.3) |
| Far 2 | 427.83 ± 36.00 (81.4) | 64.10 ± 25.48 (54.9)* | 911.65 ± 30.15 (92.7) |
| Far 20 | 437.00 ± 49.48 (83.1) | 68.22 ± 21.67 (58.5)* | 914.57 ± 43.90 (92.9) |
| Far 40 | 450.57 ± 33.72 (85.7)** | 92.37 ± 41.29 (79.2)* | 889.97 ± 23.31 (90.4) |

^{##}p < 0.05 against 5M Sham
*p < 0.005 against 5M OVX

In the hPTH administration group, both of the whole bone densities and the cancellous bone densities were higher than those in control OVX group. Significant recovery of the bone mass was observed. Particularly, the cancellous bone density was largely increased.

As shown in FIG. 11B, the cancellous bone density was dose-dependently increased in the fargesin administration group. As compared to the control OVX group, it was significantly increased in fargesin administration group. The cancellous bone density of 90% MeOH fraction administration group was higher than that of Far 2 group, and it indicated the possibility of synergic effects with chemical compounds included in the fraction other than fargesin.

### (4-3) Effect on the Cortical Bone

The cortical bone mineral content and the cortical bone density were significantly decreased in the Control OVX group compared to that of the Sham OVX group as the same as those of the whole bone and the cancellous bone. The cortical bone content was remarkably decreased. Differently from the cases of the whole bone and the cancellous bone, the bone content was not restored by the administration of PTH. It was also not significantly restored by fargesin administration or 90% MeOH fraction administration (see Table 9).

TABLE 9

| | | | | Perimeter of |
|---|---|---|---|---|
| | | Bone | Perimeter of | cortical bone |
| | Cross section | thickness | cortical bone | lining |
| | area of cortical | of cortical | adventitia | membrane |
| | bone (mm²) | bone(mm) | (mm) | (mm) |
| 5M Sham | 1.98 ± 0.18 | 0.27 ± 0.03 | 8.12 ± 0.19 | 6.40 ± 0.37 |
| 5M OVX | 1.36 ± 0.15 | 0.18 ± 0.03 | 8.07 ± 0.37 | 6.93 ± 0.47 |
| PTH | 1.69 ± 0.25 | 0.22 ± 0.03 | 8.37 ± 0.26 | 6.98 ± 0.34 |
| 90% MeOH | 1.40 ± 0.19 | 0.19 ± 0.03 | 7.85 ± 0.17 | 6.64 ± 0.19 |
| Far 2 | 1.46 ± 0.23 | 0.20 ± 0.03 | 8.02 ± 0.26 | 6.79 ± 0.19 |
| Far 20 | 1.48 ± 0.18 | 0.20 ± 0.03 | 8.04 ± 0.55 | 6.78 ± 0.68 |
| Far 40 | 1.56 ± 0.19* | 0.21 ± 0.02* | 8.14 ± 0.39 | 6.83 ± 0.38 |

*p < 0.05 against 5M OVX

As compared to Sham OVX group, the boundary length of cortical bone adventitia was slightly decreased in the Control OVX group. However, that of the cortical bone endosteum was significantly increased. In contrast, both of the boundary length of the cortical bone adventitia and the cortical bone endosteum were significantly increased in PTH administration group. The same trends were shown in fargesin administration group.

Osteogenesis mainly occurs in the bone adventitia side, and the decrease of the cortical bone mass was appears as extension of Haversian canal (porous formation in the cortical bone). Thus, boundary length of the cortical bone adventitia reflects the osteogenesis (Biomedical Engineering vol. 44, No. 4: 517-521, 2006, ibid. vol. 44, No. 4: 490-502, 2006), and the increase of the length of the cortical bone endosteum reflects the bone resorption. Therefore, it was shown that the bone resorption was increased and the osteogenesis was decreased in the OVX operation group. In contrast, there was the trend that both of the osteogenesis and bone resorption were increased together in PTH administration. It was considered that the bone mass was increased because of dominant osteogenesis. In fargesin administration group, there was the trend that both of the osteogenesis and bone resorption were dose-dependently increased.

On the other hand, as shown in Table 9, the cancellous bone density was largely increased in 90% MeOH fraction administration group, however, both of the osteogenesis and the bone resorption were not increased.

### (4-4) Bone Strength (Polar Coordinates Torsion Strength)

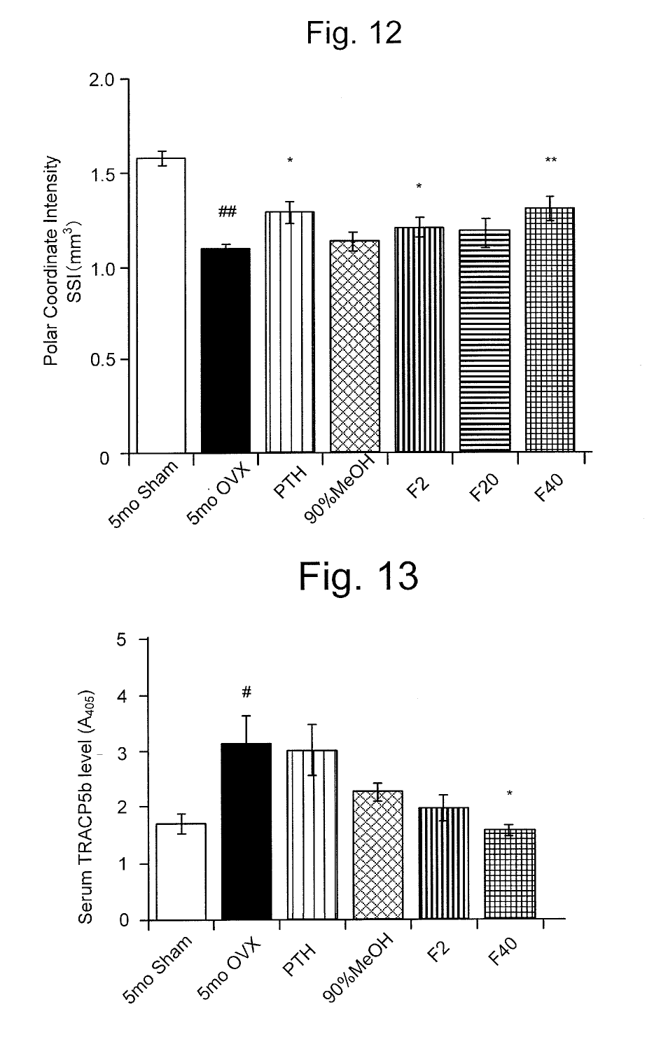

Bone strength in the Control OVX group was significantly decreased as compared to that in the Sham OVX group. The bone strengths were significantly increased in both of the PTH administration group and the fargesin administration group (2 mg and 40 mg administration groups) as compared to that of the Control OVX group. The bone strength in the 90% MeOH fraction administration group was higher than that in the Control OVX group; however, there was no significant difference between them (see FIG. 12).

### (4-5) Bone Metabolism Marker

It was known that tartrate-resistant acid phosphatase (TRACP), as a bone metabolism marker, was classified into TRACP 5a and TRACP 5b; TRACP 5a was induced from inflammatory macrophages and TRACP 5b was induced from osteoclast cells, respectively. Circadian change of the TRACP 5b level was low, and it was not affected by nutritional support. Therefore, it was known that the secreted TRACP 5b level indicates the number of osteoclast cells rather than its activity (Name of literatures: Alatalo S L, et al, Clin Chem, 46:1751-1754 (2000)., Alatalo S L, et al, J Bone Miner Res, 18:134-139 (2003)., Chu P, et al, Am J Kidney Dis, 41:1052-1059 (2003)., Alatalo S L, et al, Clin Chem, 50:883-890 (2004).).

Since TRACP 5b in the sera of the Control OVX group showed remarkably higher level compared to that of the Sham OVX group, it was considered that osteoclast cells had higher activity and large cell number in the Control OVX group (see FIG. 13)

Large fluctuation of TRACP 5b value is not seen in hPTH administration group as compared to OVX group, indicating interrelated perimeters of the cortical bone lining membranes. TRACP 5b value in 90% MeOH fraction administration is higher than that in Far 2 group, indicating interrelated perimeters of the cortical bone lining membranes. Serum TRACP5b in Fargesin administration group is decreased, showing concentration dependent and is significantly decreased in 40 mg administration group. Thus, it shows that Fargesin restrains the osteoclast from activating and increasing the number, which is the same result as RAW264.7 (FIG. 13).

Compared to the OVX group, the level of TRACP 5b did not show major alteration in hPTH administration group; however, it showed the correlation with the length of the cortical bone endosteum. TRACP 5b level in the 90% MeOH fraction administration group was higher than that of Far2 group, and it showed the correlation with the length of the cortical bone endosteum. In fargesin administration group, the level of TRACP5b in the sera was dose-dependently decreased, and it was significantly decreased in the 40 mg administration group. As a result, it was demonstrated that fargesin inhibited the activity of the osteoclast cells and the cell number, similarly to the case in which RAW264.7 was employed (see FIG. 13).

As mentioned above, it was confirmed that fargesin increased the bone densities of the whole bone and the cancellous bone. The measurement results of the boundary length of the cortical bone adventitia, the cortical bone endosteum, and serum TRACP5b activity indicated that fargesin inhibited the osteoclast cell activity and cell numbers, thereby decreasing the capability of the bone resorption to have beneficial osteogenesis effects.

### Example 7

### Evaluation of Efficiency with Fargesin for an Osteopenia Mouse Caused by sRANKL Administration

(1) Test animal

C57BL/6NCrlCrlj mice (6-week-age, female) were purchased from Oriental Yeast Co., Ltd. and kept to be acclimatized under the conditions of 12 hour light/dark cycle, 23±3 degree centigrade, and humidity 55±5% for 7 days. Two mice were in a cage, and the animal bedding was changed twice a week, and all fresh animal bedding was used at all times. As the feed, CRF-1 (Oriental Yeast Co., Ltd.), and deionized water as the drinking water were freely taken.

### (2) Test Methods-Administration of Test Substances

After the acclimatization, these mice were randomly divided into 4 groups (8 mice per group), sRANKL administration group and fargesin administration groups. Fargesin administration groups had 0.2 mg/kg/day administration group (it is referred to as “Far 0.2”, hereinbelow), 2 mg/kg/day administration group (it is referred to as “Far 2” herein below), and 20 mg/kg/day administration group (it is referred to as “Far 20” herein below). sRANKL (1 mg/kg/day, i.p) was administered to all mice on the first day and the second day from test start to cause osteopenia experimentally.

From the 4^{th} day to 13^{th} day after the test start, distilled water or the test compounds shown in the following Table 10 were daily administered to the mice in each group.

TABLE 10

| | Name of | Test solution | |
|---|---|---|---|
| Groups administrated | groups | administrated | Route |
| Control group | RANKL | RANKL water solution | p.o. |
| | | (1 mg/kg/day) | |
| hPTH administrated | Human PTH | hPTH(1-34) water solution | s.c. |
| group | (1-34) | (80 μg/kg/day) | p.o. |
| Test substance | Fargesin 0.2 | TD solution of fargesin | p.o. |
| administrated group | | (0.2 mg/kg/ day) | |
| | Fargesin 2 | TD solution of fargesin | p.o. |
| | | (2 mg/kg/day) | |
| | Fargesin 20 | TD solution of fargesin | p.o. |
| | | (20 mg/kg/day) | |

### (3) Study of Effects on the Bone

### (3-1) Preparation of the Bone

After the administration period was finished and the experiment was completed, the femurs were removed in the same way as employed in Example 6 to be excised to evaluate the bone density. Body weight of the test animals and feed intake by the test animals were measured twice a week during the administration period.

The experiments were performed as the same as done in Example 6, right femur chosen from the obtained femurs was used for measurement of the bone density.

### (3-2) Measurements of the Bone Density, the Bone Strength, and the Change of the Tissue Weight

Results from the measurements of the body weights of each group did not show deviation depending on the groups.

The whole bone density, the cancellous bone density, the cortical bone density, the boundary length of the cortical bone adventitia, the length of the cortical bone endosteum, and the bone strength were measured at the site of −0.6 mm from the growth plate of the bone (see FIG. 3A).

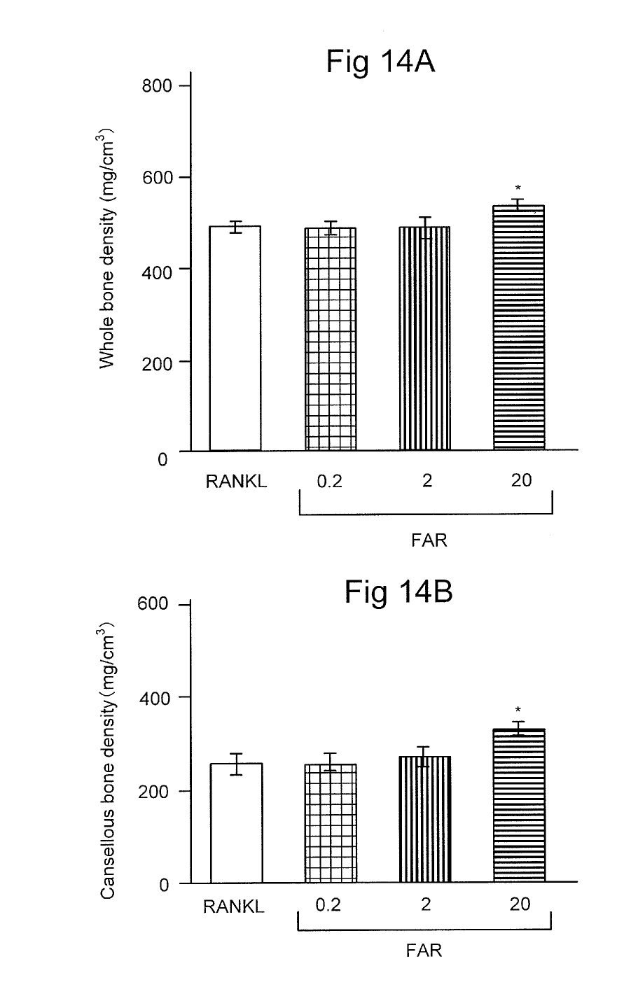

As shown in FIGS. 14A and 14B, the whole bone density was significantly increased depending on the fargesin dose. The cancellous bone density was also significantly increased, depending on the fargesin dose.

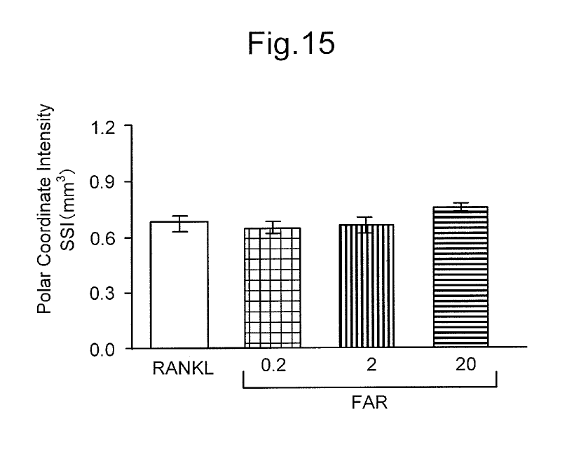

The bone strength was evaluated by using Polar coordinates strength (SSI). As shown in FIG. 15, there was the trend that the bone strength was improved, depending on fargesin dose.

As mentioned above, it was demonstrated that fargesin has the effects on the bone loss of the young age animals caused by RANKL administration thorough dose-dependent increase of the bone density.

TABLE 11

| | | Bone density | Bone density |
|---|---|---|---|
| | | of the | of the |
| | Bone density of whole | cancellous bone | cortical bone |
| | bone (mg/cm³) | (mg/cm³) | (mg/cm³) |
| RANKL | 486.60 ± 14.10 (100.0) | 259.91 ± 23.32 | 807.66 ± 5.59 |
| | | (100.0) | (100.0) |
| Far 0.2 | 484.33 ± 11.09 (99.5) | 261.64 ± 17.92 | 797.40 ± 6.12 |
| | | (100.7) | (98.7) |
| Far 2 | 487.30 ± 16.36 (99.5) | 270.30 ± 20.64 | 798.60 ± 4.56 |
| | | (104.0) | (98.9) |
| Far 20 | 532.69 ± 8.06 (110)* | 336.25 ± 13.50 | 796.33 ± 3.89 |
| | | (129.4)* | (98.6) |

*p < 0.05 against RANKL

### Blending Example

Bleeding examples of the food comprising fargesin or 90% MeOH fraction are shown in below. Each blending example may be used for producing the functional food or the health foods.

### Blending Example 1

### Chewing Gum

TABLE 12

| Compositions | wt % |
|---|---|
| Sugar | 53.0 |
| Gum base | 20.0 |
| Glucose | 10.0 |
| Starch syrup | 16.0 |
| Spice | 0.5 |
| Composition of the | 0.5 |
| present invention | |
| Total | 100.0 |

### Blending Example 2

### Gumi Candy

TABLE 13

| Composition | wt % |
|---|---|
| Reduced sugar syrup | 40.0 |
| Granulated sugar | 20.0 |
| glucose | 20.0 |
| gelatin | 4.6 |
| Water | 9.7 |
| Orange fruit juice | 4.0 |
| Orange flavor | 0.7 |
| Composition of the | 1.0 |
| present invention | |
| Total | 100.0 |

### Blending Example 3

### Candy

TABLE 14

| Composition | wt % |
|---|---|
| Sugar | 50.0 |
| Starch syrup | 33.0 |
| Water | 14.4 |
| Organic acid | 2.0 |
| flavoring | 0.2 |
| Composition of the | 0.4 |
| present invention | |
| Total | 100.0 |

### Blending Example 4

### Yogurt (Hard & Soft)

TABLE 15

| Composition | Wt % |
|---|---|
| Milk | 41.5 |
| Powdered skim milk | 5.8 |
| Sugar | 8.0 |
| Agar | 0.15 |
| gelatin | 0.1 |
| lactobacillus | 0.005 |
| Composition of the | 0.4 |
| present invention | |
| flavoring | trace |
| Water | residue |
| Total | 100.0 |

### Blending Example 5

### Soft Capsule

TABLE 16

| Composition | wt % |
|---|---|
| Brown rice germ oil | 87.0 |
| Emulsifier | 12.0 |
| Composition of the | 1.0 |
| present invention | |
| Total | 100.0 |

### Blending Example 6

### Coffee Beverage

TABLE 17

| Composition | Wt. % |
|---|---|
| Roasted coffee beans | 6.0 |
| Sugar | 6.0 |
| Sodium bicarbonate | 0.2 |
| Emulsifier | 0.15 |
| Composition of the | 1.0 |
| present invention | |
| Water | residue |
| Total | 100.0 |

### Blending Example 7

### Coffee Beverage (Powder)

TABLE 18

| Composition | Wt. % |
|---|---|
| Instant coffee | 90.0 |
| Skim milk | 7.0 |
| Composition of the | 3.0 |
| present invention | |
| Total | 100.0 |

### Blending Example 8

### Refreshment

| Composition | Wt. % |
|---|---|
| Fructose glucose syrup | 30.0 |
| Emulsifier | 0.5 |
| Flavor | Appropriate |
| | amount |
| Composition of the | 1.0 |
| present invention | |
| Purified water | residue |
| Total | 100.0 |

### Blending Example 9

### Confectionary Tablet

TABLE 20

| Composition | Wt. % |
|---|---|
| Sugar | 76.4 |
| Glucose | 19.0 |
| Sucrose fatty acid ester | 0.2 |
| Composition of the invention | 0.5 |
| Purified Water | residue |
| Total | 100.0 |

### (Pharmaceutical Preparation)

Next, the pharmaceutical preparation comprising the composition was shown in below. However, the present invention is not limited to the examples.

### (Pharmaceutical Preparation 1 Tablet)

TABLE 21

| Component | Usage (g) |
|---|---|
| Composition 1 | 100 |
| Mannitol | 123 |
| Starch | 33 |
| Crospovidone | 12 |
| Microcrystalline cellulose | 30 |
| Magnesium Stearate | 2 |

The compositions were respectively weighted, and homogenously mixed. Then, 300 mg of the mixture was compressed to form a tablet.

### (Pharmaceutical Preparation 2 Hard Capsule)

TABLE 22

| Component | Usage (g) |
|---|---|
| Composition 1 | 40 |
| lactose | 150 |
| starch | 70 |
| Polyvinylpyrrolidone | 5 |
| Crystalline cellulose | 35 |

The compositions were respectively weighted, and homogenously mixed. Then, 300 mg of the mixture was filled with a hard capsule. Here, the composition 1 is composed of either fargesin or 90% MeOH fraction, and lactose 1:1. Note that the composition employed in the pharmaceutical preparations 3 to 6 are the same as the composition 1.

### (Pharmaceutical Preparation 3 Soft Capsule)

TABLE 23

| Component | Usage (g) |
|---|---|
| Composition 1 | 100 |
| Tocopherol | 0.2 |

The compositions were respectively weighted, and homogenously mixed. Then, 100 mg of the mixture was filled with a soft capsule.

### (Pharmaceutical Preparation 4 Granule Agent)

TABLE 24

| Component | Usage (g) |
|---|---|
| Composition 1 | 200 |
| Lactose | 450 |
| Corn starch | 300 |
| Hydroxypropyl cellulose | 50 |
| Crystalline cellulose | 35 |

The compositions were respectively weighted, and homogenously mixed to produce granule agent by using conventional method.

### (Pharmaceutical Preparation 5 Syrup Agent)

TABLE 25

| Component | Usage (g) |
|---|---|
| Composition 1 | 2 |
| Saccharin | 0.6 |
| Sugar | 30 |
| glycerin | 5 |
| Seasoning | 0.1 |
| 96% ethanol | 10.42 |
| Purified water | Amount to final volume 100 ml |

The above-mentioned compositions were weighed, and both sugar and saccharin were dissolved in 60 ml of distilled water for injection. Then, the composition 2 dissolved in glycerin and ethanol and a solution of seasonings were added to have a mixture. Distilled water was added to the mixture to become final amount to 100 mL to prepare a syrup agent.

### (Pharmaceutical Preparation Granule)

TABLE 26

| Component | Usage (g) |
|---|---|
| Composition 1 | 100 |
| Calcium silicate | 100 |

### INDUSTRIAL APPLICABILITY

The present invention is useful in the field of production and development for pharmaceutical preparations, functional food, health food and the like.

1. A pharmaceutical composition for promoting osteoblast differentiation comprising at least a substance selected from the group consisting of a compound shown in the following chemical formula (I), a pharmacologically acceptable salt thereof, a pharmacologically acceptable hydrate thereof, and a pharmacologically acceptable glycoside thereof. 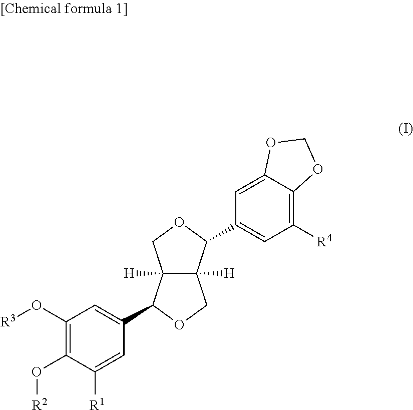
(In the formula, R¹ and R⁴ independently show one of functional group selected from the group consisting of a hydrogen atom, alkyl group having a carbon number 1 to 3, hydroxyl group, alkoxy group having the carbon number 1 to 3; R² and R³ independently show one of functional group selected from the group consisting of alkyl group having a carbon number 1 to 3.)

2. The pharmaceutical composition for promoting osteoblast differentiation according to the claim 1, comprising at least a substance selected from the group consisting of a compound shown in the following chemical formula (II), a pharmacologically acceptable salt thereof, a pharmacologically acceptable hydrate thereof, and a pharmacologically acceptable glycoside thereof.

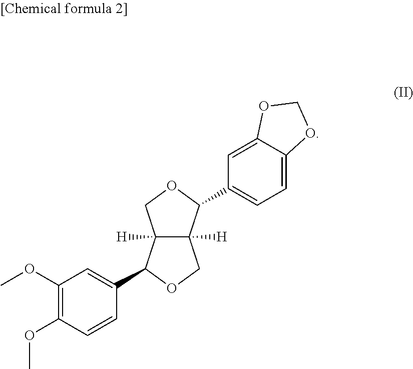 [SMILES: COc1ccc([C@@H]2OC[C@H]3[C@@H]2CO[C@@H]3c2ccc3c(c2)OCO3)cc1OC]

3. A pharmaceutical composition for promoting osteoblast differentiation comprising an extract fraction obtained from one organ selected from the group consisting of a flower bud, leaf, cortex and xylem of Magnoliaceae plant, of which fraction containing the compound shown in the above-mentioned formula (II).

4. The pharmaceutical composition for promoting osteoblast differentiation according to the claim 3, wherein the organ selected from the group consisting of the flower bud, leaf, cortex and xylem of Magnoliaceae plant is obtained from the plant selected from the group consisting of Tamushiba (Magnolia salicifolia Maximowicz), Kobushi (Magnolia kobus De Candolle, Magnolia biondii Pampanini, Magnolia sprengeri Pampanini), Hakumokuren (Magnolia heptapeta Dandy (Magnolia denudata Desrousseaux) (Magnoli-aceae), and Kitakobushi (Magnolia praecocissima var. borealis).

5. A pharmaceutical preparation comprising the pharmaceutical composition for promoting osteoblast differentiation according to the any one of the claims 1 or 3 for administrating a predetermined dose as an active ingredient.

6. The pharmaceutical preparation for promoting osteoblast differentiation according to the claim 5, wherein the predetermined dose is 10 to 350 mg/day in compound equivalent.

7. A functional food comprising the composition according to any of the claims 1 or 3.

8. The functional food according to the claim 7, wherein a content of the composition is 1 to 1,000 mg/100 g.

9. The functional food according to the claim 7, wherein the functional food is selected from the group consisting of a biscuit, wheat and coarse cereal to be supplemented to rice, noodle such as buckwheat noodle and pasta, dairy product such as cheese and yoghurt, jam, mayonnaise, processed soy product such as soy source, non-alcoholic beverage such as coffee and cocoa, alcoholic beverage such as medicated liquor, snacks such as candy and chocolate, confectionery such as rice cracker and youkan.

10. A healthy food comprising the composition for promoting osteoblast differentiation according to any one of the claims 1 or 3.

11. The healthy food according to the claim 10, wherein a content of the composition is 1 to 1,000 mg/100 g.

12. The healthy food according to the claim 10, wherein the functional food is selected from the group consisting of a biscuit, wheat and coarse cereal to be supplemented to rice, noodle such as buckwheat noodle and pasta, dairy product such as cheese and yoghurt, jam, mayonnaise, processed soy product such as soy source, non-alcoholic beverage such as coffee and cocoa, alcoholic beverage such as medicated liquor, snacks such as candy and chocolate, confectionery such as rice cracker and youkan.
